(19) United States
(12) Patent Application Publication
(10) Pub. No.: US 2016/0015710 A1
(43) Pub. Date: Jan. 21, 2016

(54) Methods of Treating Multiple Myeloma and Resistant Cancers

(71) Applicant: Prolexys Pharmaceuticals, Inc., Columbia, MD (US)

(72) Inventors: Paul B. Robbins, Park City, UT (US); Sudhir R. Sahasrabudhe, Sandy, UT (US)

(21) Appl. No.: 14/831,798

(22) Filed: Aug. 20, 2015

Related U.S. Application Data
(63) Continuation of application No. 14/096,571, filed on Dec. 4, 2013, now abandoned.
Application No. 14/096,571 is a continuation of application No. 12/665,676, filed on Jan. 26, 2011, now abandoned.
(60) Provisional application No. 60/937,238, filed on Jun. 25, 2007.

(51) Int. Cl. A61K 31/517; A61K 31/704; A61K 38/05; A61K 45/06; A61K 31/454

(52) U.S. Cl. CPC A61K 31/517; A61K 45/06; A61K 31/454; A61K 38/05; A61K 31/704

(57) ABSTRACT

Erastin analogs are useful in treating various cancers, particularly multiple myeloma. Erastin analogs are also useful in treating cancers that are resistant to other anticancer agents. This abstract is intended as a scanning tool for purposes of searching in the particular art and is not intended to be limiting of the present invention.

### RELATED APPLICATION

This application claims the benefit of U.S. Provisional Application No. 60/937,238, filed Jun. 25, 2007, the contents of which are incorporated herein by reference in their entirety.

### BACKGROUND OF THE INVENTION

Many drugs administered to treat a disease are targeted against general differences between a diseased cell and a normal cell. For example, paclitaxel, which is used to treat ovarian and breast cancer and inhibits microtubule function, is thought to exhibit tumor cell specificity based on the greater rate of proliferation of tumor cells relative to normal cells (Miller and Ojima, Chem. Rec. 1:195-211, 2002). However, despite this consensus view, paclitaxel's in vitro activity varies widely across tumor cell lines (Weinstein et al., Science 275:343-349, 1997), indicating that genetic factors can modify sensitivity of tumor cells to paclitaxel and that the responsiveness of tumor cells is not simply determined by their rate of proliferation.

Molecularly targeted therapeutics represent a promising new approach to anti-cancer drug discovery (Shawver et al., Cancer Cell 1:117-23, 2002). Using this approach, small molecules are designed to inhibit directly the very oncogenic proteins that are mutated or overexpressed in specific tumor cell types. By targeting specific molecular defects found within tumor cells, this approach may ultimately yield therapies tailored to each tumor's genetic makeup. Two recent examples of successful molecularly targeted anti-cancer therapeutics are Gleevec (imatinib mesylate), an inhibitor of the breakpoint cluster region-abelsen kinase (BCR-ABL) oncoprotein found in Philadelphia chromosome-positive chronic myelogenous leukemia (Capdeville et al., Nat Rev Drug Discov 1:493-502, 2002) and Herceptin (trastuzumab), a monoclonal antibody targeted against the HER2/NEU oncoprotein found in metastatic breast cancers (Mokbel and Hassanally, Curr Med Res Opin 17:51-9, 2001).

A complementary strategy involves searching for genotype-selective anti-tumor agents that become lethal to tumor cells only in the presence of specific oncoproteins or in the absence of specific tumor suppressors. Such genotype-selective compounds might target oncoproteins directly or they might target other critical proteins involved in oncoprotein-linked signaling networks. Compounds that have been reported to display synthetic lethality include (i) the rapamycin analog CCI-779 in myeloma cells lacking PTEN (Shi et al., Cancer Res 62:5027-34, 2002), (ii) Gleevec in BCR-ABL-transformed cells (Druker et al., Nat Med 2:561-6, 1996) and (iii) a variety of less well-characterized compounds (Stockwell et al., Chem Biol 6:71-83, 1999; Torrance et al., Nat Biotechnol 19:940-5, 2001).

Despite the research discussed above, there remains a significant need to develop and/or identify compounds that selectively target tumor cells.

### SUMMARY OF THE INVENTION

A number of compounds/agents/drugs useful for treating or preventing various cancers (e.g., multiple myeloma, including drug-resistant variants) in an individual, such as a human in need of treatment or prevention, have been identified. As used herein, the terms “agent” and “drug” are used interchangeably; they can be compounds or molecules. Suitable agents can have the recited activity in the existing form or after complete or partial metabolism.

The compounds of the invention can be formulated with a pharmaceutically acceptable carrier as pharmaceutical compositions for treatment of the conditions disclosed herein.

The present invention further provides packaged pharmaceuticals. In one embodiment, the packaged pharmaceutical comprises: (i) a therapeutically effective amount of a compound disclosed herein; and (ii) instructions and/or a label for administration of the agent for the treatment of patients having an indication disclosed herein. The instruction or label may be stored on an electronic medium such as CD, DVD, floppy disk, memory card, etc., which may be readable by a computer.

The present invention further provides use of a compound disclosed herein in the manufacture of a medicament for the treatment of an indication disclosed herein.

In certain embodiments, the methods of the invention further comprise conjointly administering one or more agents, such as chemotherapeutic agents that typically kill the cells through an apoptotic mechanism. Agents suitable for use in reducing the growth rate of a tumor and in treating a patient suffering from cancer include, but are not limited to, small organic molecules, peptides, proteins, peptidomimetics, nucleic acids, antibodies and combinations thereof.

It is contemplated that all embodiments of the invention can be combined with one or more other embodiments, even those described under different aspects of the invention.

### BRIEF DESCRIPTION OF THE DRAWINGS

FIG. 1 shows the in vitro effect of Compound 5 of Group I on various multiple myeloma cells. The cells towards the top of the figure are the most sensitive to the compound.

FIG. 2 shows the effect of Compound 5 of Group I in the presence of HS-5 stroma.

FIG. 3 shows the effect of Compound 5 of Group I on CD 138+ cells of multiple myeloma patients.

FIG. 4 shows the effect of pulses of Compound 5 of Group I on OPM-2 multiple myeloma cells.

FIG. 5 shows the effect of pulses of Compound 5 of Group I on NCI-H929 cells.

FIG. 6 shows the correlation between N,K-ras mutations and sensitivity to Compound 5 of Group I.

FIG. 7 shows genes whose expression was significantly changed after cells were exposed to Compound 5 of Group I.

FIG. 8 shows a schematic outline of in vivo mouse experiments with compounds of the invention.

FIG. 9 shows the cumulative survival of mice injected with human multiple myeloma cells and treated with Compound 5 of Group I, as compared to control mice not treated with Compound 5.

FIG. 10 shows in vivo imaging of MM.1S* cells in mice treated with Compound 5 of Group I (100 mg/kg), VELCADE (0.5 mg/kg) or nothing.

FIG. 11A and FIG. 11B show cells which genes are relatively sensitive or insensitive to Compound 5 of Group I.

FIG. 12 shows the in vitro effect of Compound 5 of Group I on peripheral blood mononuclear cells.

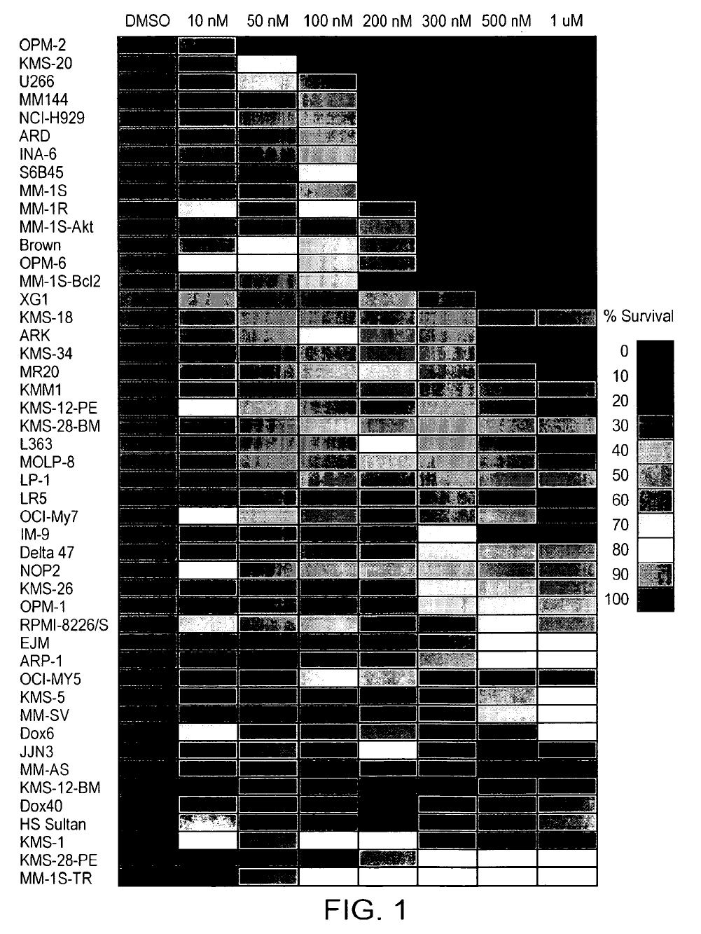

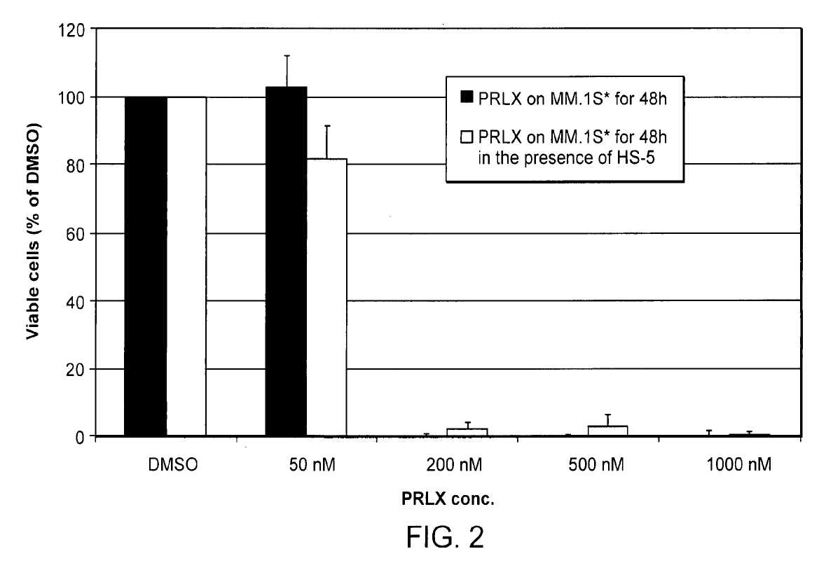

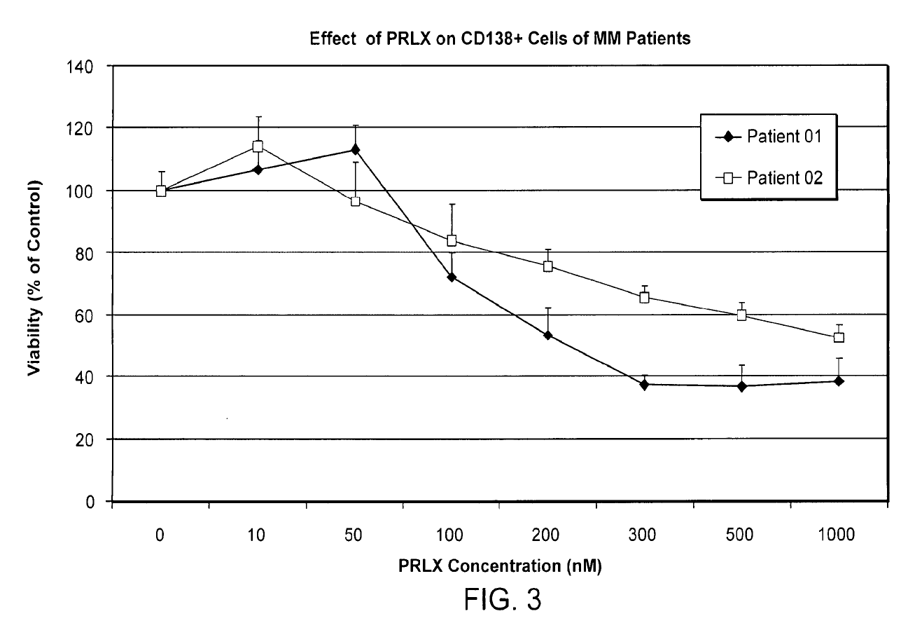

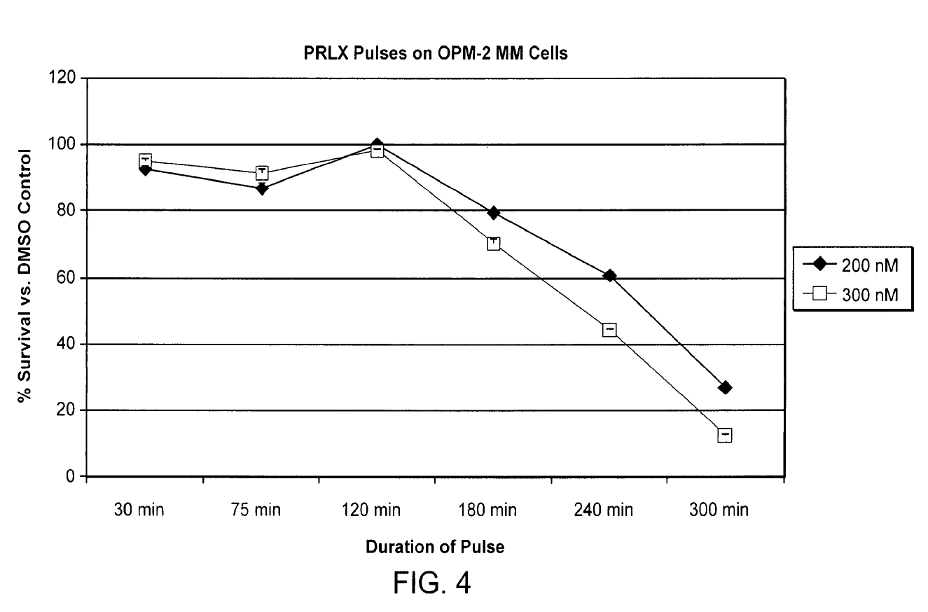

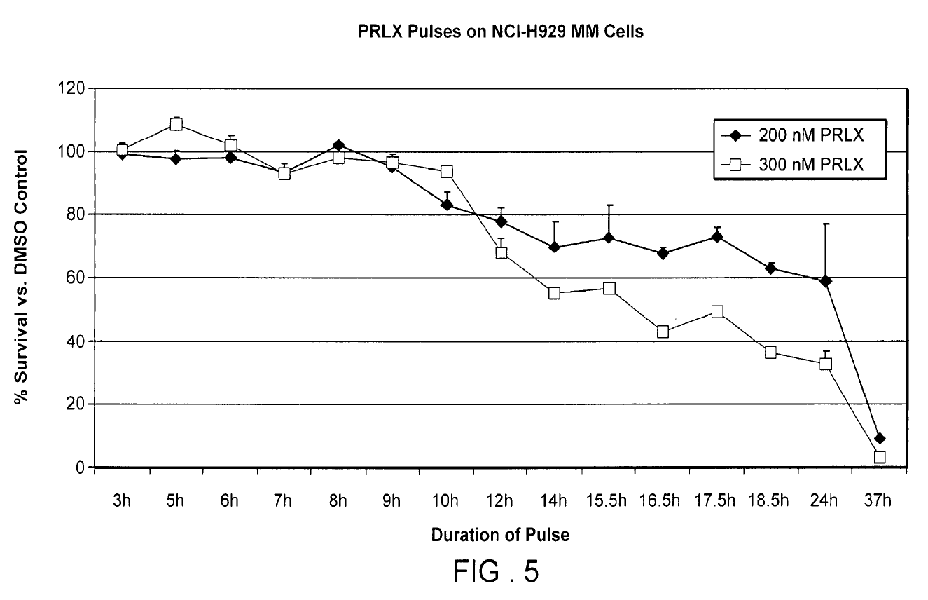

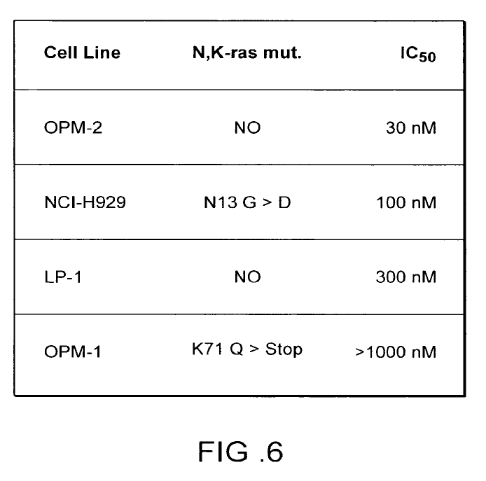

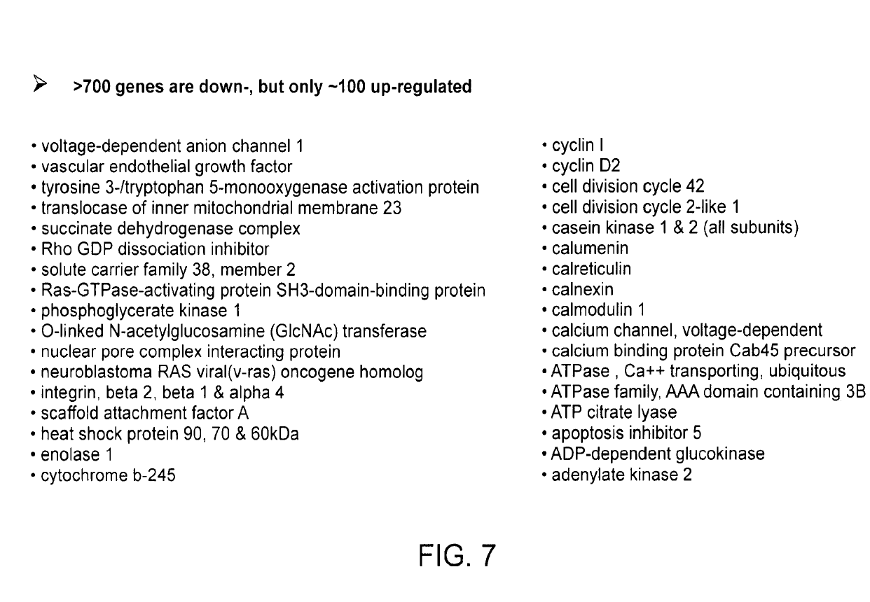

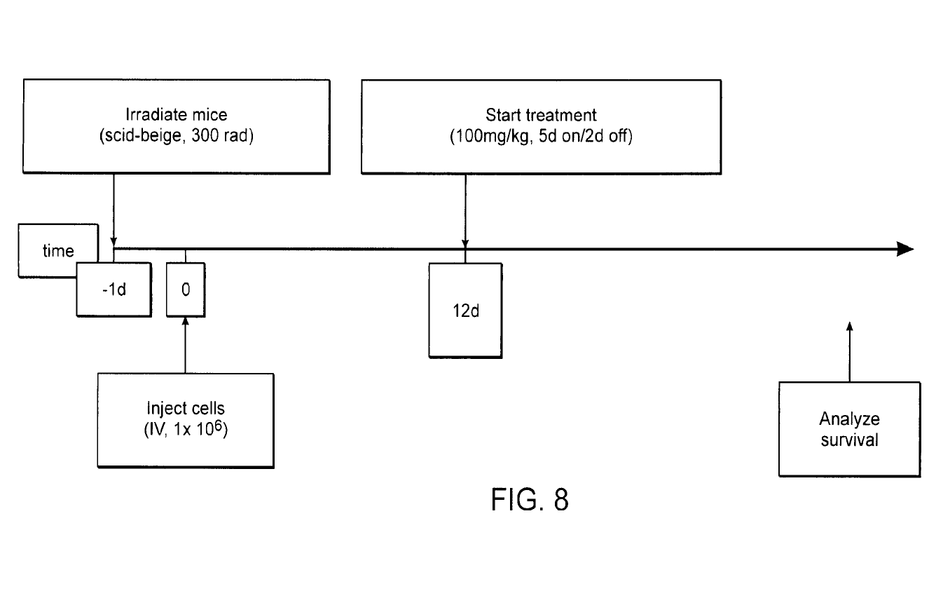

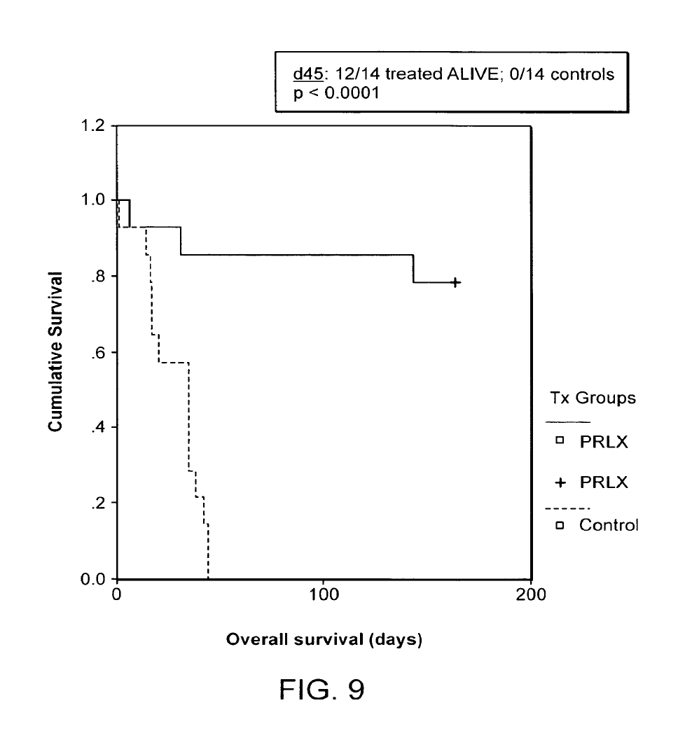

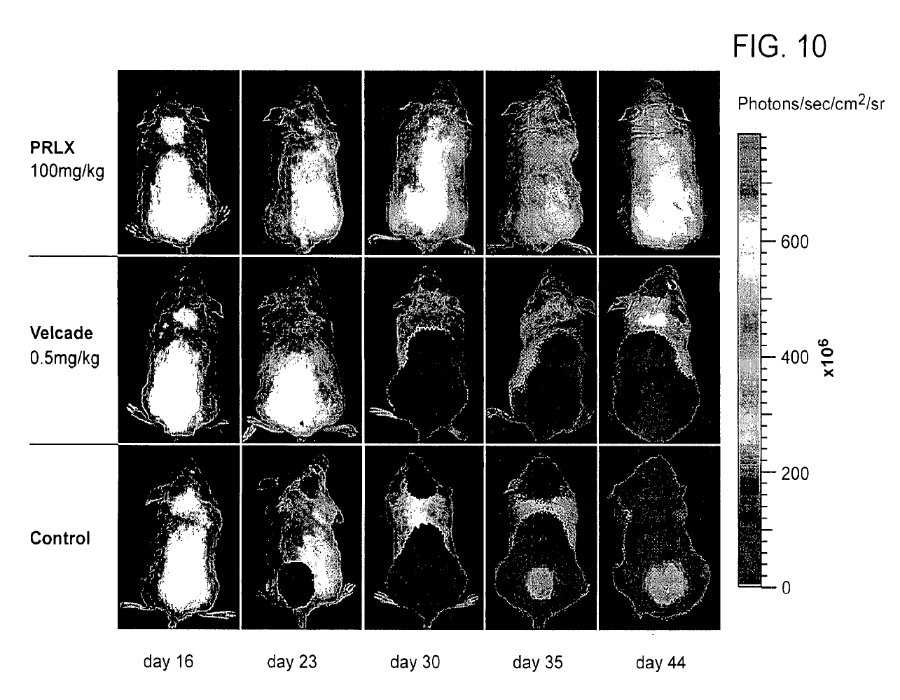

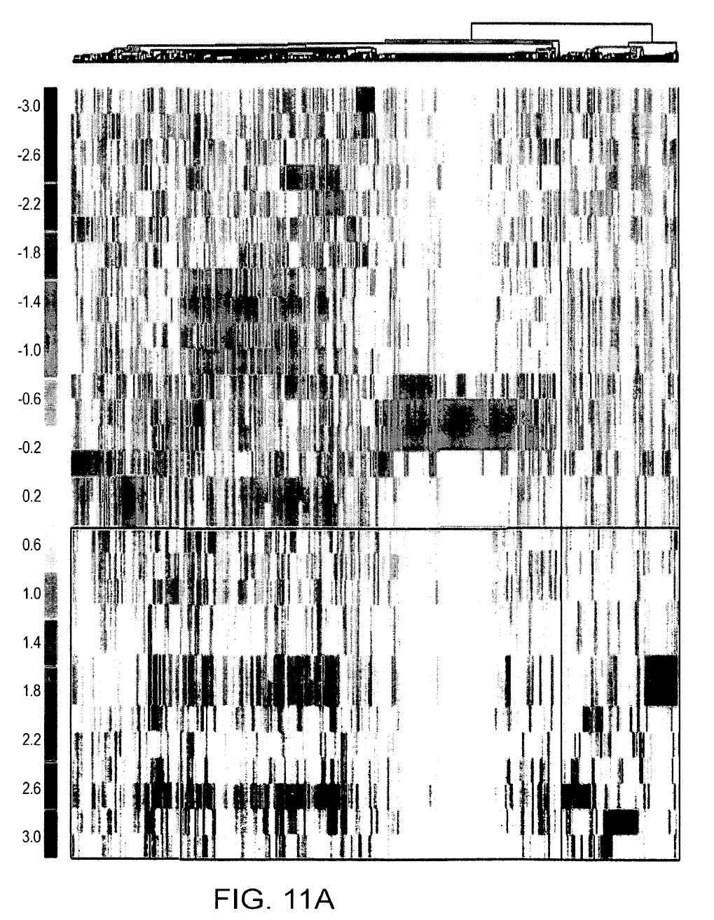

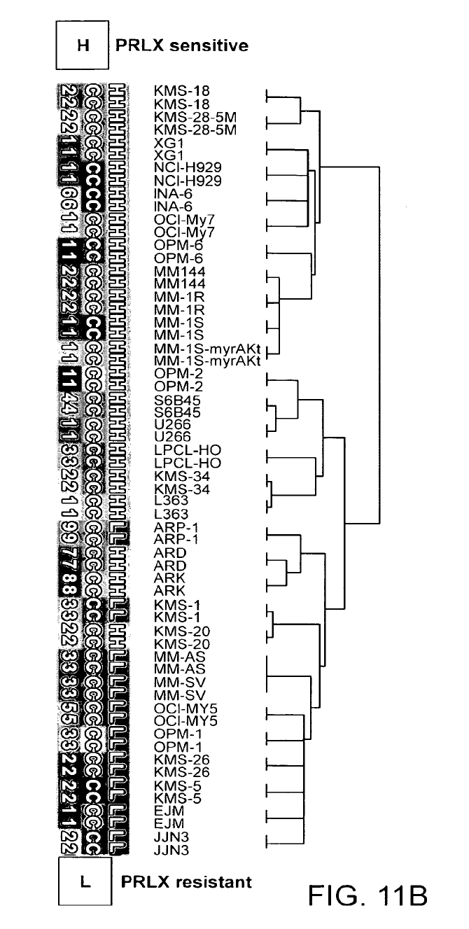

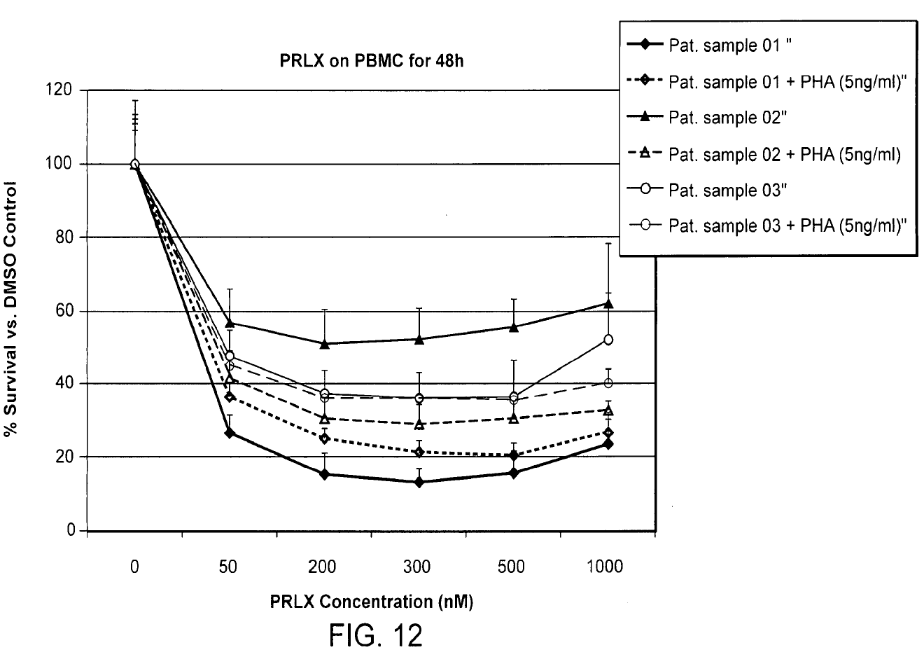

### DETAILED DESCRIPTION OF THE INVENTION

A variety of compounds are suitable for use in the methods and compositions described herein. In certain embodiments, compounds of the invention are erastin analogues, as defined in US 2006/0211683, WO 2007/076085 and WO 2007/076087 the contents of which are incorporated herein by reference. Particular compounds suitable for use in the invention are designated as Groups I-III below. The substituents on such compounds only apply to the compounds of the same group. Methods of preparing these compounds can be found, for example, in US 2006/0211683 and in WO 2007/076085 and WO 2007/076087, the contents of which are incorporated herein by reference.

Group I

In certain embodiments, compounds suitable for use in the invention are represented by the general formula I:

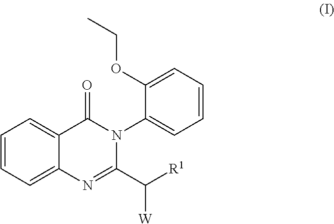

wherein:

R¹ is selected from H, —Z-Q-Z, —C₁₋₈alkyl-N(R²)(R⁴), —C₁₋₈alkyl-OR³, 3- to 8-membered carbocyclic or heterocyclic, aryl, heteroaryl, and C₁₋₄aralkyl;

R² and R⁴ are each independently for each occurrence selected from H, C₁₋₄alkyl, C₁₋₄aralkyl, aryl, heteroaryl, acyl, alkylsulfonyl, and arylsulfonyl, provided that when both R² and R⁴ are on the same N and either R² or R⁴ is acyl, alkylsulfonyl, or arylsulfonyl, then the other is selected from H, C₁₋₈alkyl, C₁₋₄aralkyl, and heteroaryl;

R³ is selected from H, C₁₋₄alkyl, C₁₋₄aralkyl, aryl, and heteroaryl;

W is selected from

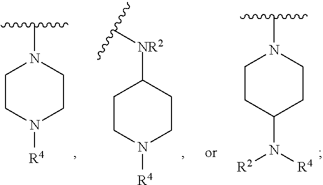

Q is selected from O and NR²; and

Z is independently for each occurrence selected from C₁₋₆alkyl, C₂₋₆alkenyl, and C₂₋₆alkynyl. When Z is an alkenyl or alkynyl group, the double or triple bond or bonds are preferably not at the terminus of the group.

In certain preferred embodiments when both R² and R⁴ are on the same N atom they are either both H or are different.

In certain embodiments, R¹ is H.

In certain embodiments, W is

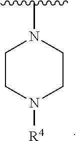

In certain embodiments, R⁴ is selected from H or substituted or unsubstituted lower alkyl.

In certain embodiments, R¹ is H, W is

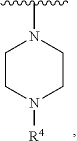

and R⁴ is selected from H or substituted or unsubstituted lower alkyl.

Exemplary compounds of formula I include:

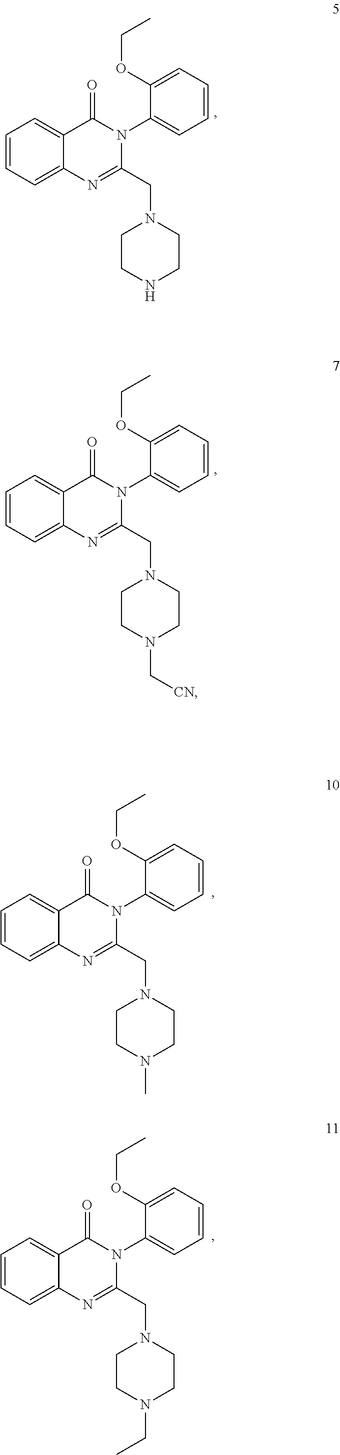

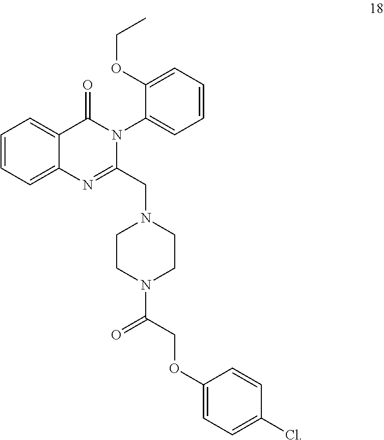 [SMILES: CCOc1ccccc1-n1c(CN2CCN(C(=O)COc3ccc(Cl)cc3)CC2)nc2ccccc2c1=O]

Additional compounds suitable for use in the invention are represented by the general formula II:

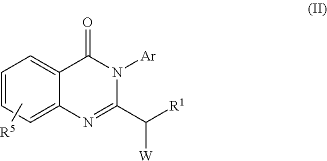

wherein:

Ar is a substituted phenyl;

R¹ is selected from H, C₁₋₈alkyl, —Z-Q-Z, —C₁₋₈alkyl-N(R²)(R⁴), —C₁₋₈alkyl-OR³, 3- to 8-membered carbocyclic or heterocyclic, aryl, heteroaryl, and C₁₋₄aralkyl;

R² and R⁴ are each independently for each occurrence selected from H, C₁₋₄alkyl, C₁₋₄aralkyl, aryl, heteroaryl, acyl, alkylsulfonyl, and arylsulfonyl, provided that when both R² and R⁴ are on the same N and either R² or R⁴ is acyl, alkylsulfonyl, or arylsulfonyl, then the other is selected from H, C₁₋₈alkyl, aryl, C₁₋₄aralkyl, aryl, and heteroaryl;

R³ is selected from H, C₁₋₄alkyl, C₁₋₄aralkyl, aryl, and heteroaryl;

R⁵ represents 0-4 substituents on the ring to which it is attached;

W is

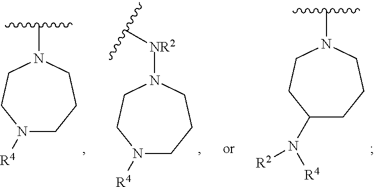

Q is selected from O and NR²; and

Z is independently for each occurrence selected from C₁₋₆alkyl, C₂₋₆alkenyl, and C₂₋₆alkynyl. When Z is an alkenyl or alkynyl group, the double or triple bond or bonds are preferably not at the terminus of the group.

In certain embodiments, R⁵ represents 1-4 substituents, such as halogen or nitro. In certain embodiments, R⁵ represents one substituent, such as halogen or nitro, especially chloro, situated para to the carbonyl of the quinazolinone ring. In other embodiments, R⁵ represents no substituents on the ring (i.e., all substituents are hydrogen atoms).

In certain embodiments, Ar is mono-substituted wherein the substituent is halogen, lower alkoxy, or lower alkyl. In certain embodiments, Ar has a substituent at the ortho position wherein the substituent is halogen, lower alkoxy, or lower alkyl. In certain embodiments, Ar is 2,6-disubstituted such that one substituent is halogen, lower alkoxy, or lower alkyl and the second substituent is halogen, lower alkoxy, or lower alkyl.

In certain embodiments, the compounds of formula II do not include those wherein the substituent on Ar is ethoxy at a position ortho to the bond to the nitrogen of the quinazolinone ring. In further embodiments, the compounds of formula II do not include those wherein Ar does not have a lower alkoxy or lower alkyl substituent ortho to the bond to the nitrogen of the quinazolinone ring.

In certain embodiments of the compounds of formula II, Ar has at least one halogen substituent. In certain embodiments, Ar has a halogen substituent in the ortho position. In preferred embodiments, the compounds of formula II include those wherein Ar is a 2,6-disubstituted phenyl ring wherein the substituents are halogen atoms.

Exemplary compounds of formula II include:

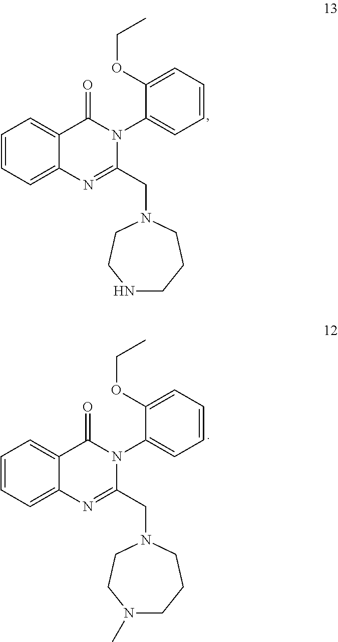 [SMILES: CCOc1ccccc1-n1c(CN2CCCNCC2)nc2ccccc2c1=O ; CCOc1ccccc1-n1c(CN2CCCN(C)CC2)nc2ccccc2c1=O]

Other compounds suitable for use in the invention are represented by the general formula III:

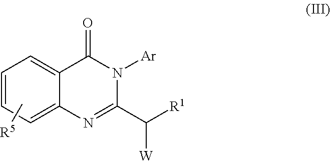

wherein:

Ar is a substituted or unsubstituted phenyl;

R¹ is selected from H, C₁₋₈alkyl, —Z-Q-Z, —C₁₋₈alkyl-N(R²)(R⁴), —C₁₋₈alkyl-OR³, 3- to 8-membered carbocyclic or heterocyclic, aryl, heteroaryl, and C₁₋₄aralkyl;

R² and R⁴ are each independently for each occurrence selected from H, C₁₋₄alkyl, C₁₋₄aralkyl, aryl, heteroaryl, acyl, alkylsulfonyl, and arylsulfonyl, provided that when both R² and R⁴ are on the same N atom and either R² or R⁴ is acyl, alkylsulfonyl, or arylsulfonyl, then the other is selected from H, C₁₋₈alkyl, aryl, C₁₋₄aralkyl, and heteroaryl;

R³ is selected from H, C₁₋₄alkyl, C₁₋₄aralkyl, aryl, and heteroaryl;

R⁵ represents 0-4 substituents on the ring to which it is attached;

W is selected from

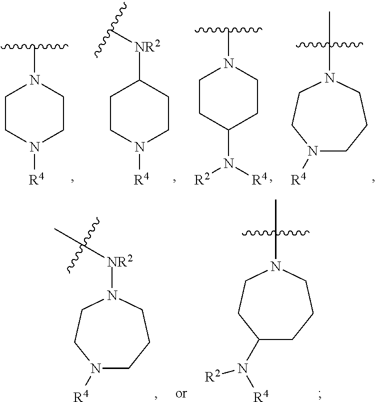

Q is selected from O and NR²; and

Z is independently for each occurrence selected from C₁₋₆alkyl, C₂₋₆alkenyl, and C₂₋₆alkynyl. When Z is an alkenyl or alkynyl group, the double or triple bond or bonds are preferably not at the terminus of the group.

In certain embodiments, R² and R⁴ are either both H or are different.

In certain embodiments, R⁵ represents from 1-4 substituents on the ring to which it is attached, such as halogen or nitro. In certain embodiments, R⁵ represents one substituent, such as halogen or nitro, especially chloro, situated para to the carbonyl of the quinazolinone ring. In other embodiments, R⁵ represents no substituents on the ring (i.e., all substituents are hydrogen atoms).

In certain embodiments, the compounds of formula III do not include those wherein the substituent on Ar is ethoxy at a position ortho to the bond to the nitrogen of the quinazolinone ring. In further embodiments, the compounds of formula III do not include those wherein Ar does not have a lower alkoxy or lower alkyl substituent ortho to the bond to the nitrogen of the quinazolinone ring.

In preferred embodiments of formula III, Ar is a substituted phenyl. In certain embodiments of the compounds of formula III, Ar has at least one halogen substituent. In certain embodiments, Ar has a halogen substituent in the ortho position. In preferred embodiments, the compounds of formula III include those wherein Ar is a 2,6-disubstituted phenyl ring wherein the substituents are halogen atoms.

Exemplary compounds of formula III include:

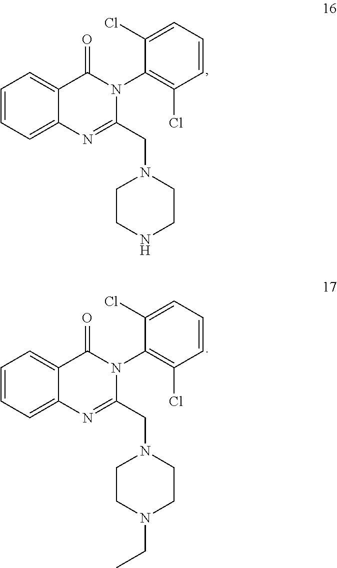 [SMILES: O=c1c2ccccc2nc(CN2CCNCC2)n1-c1c(Cl)cccc1Cl ; CCN1CCN(Cc2nc3ccccc3c(=O)n2-c2c(Cl)cccc2Cl)CC1]

Further compounds suitable for use in the invention are represented by the general formula IV:

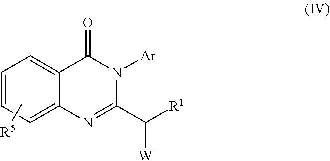

wherein:

Ar is substituted or unsubstituted phenyl;

R¹ is C₁₋₈alkyl;

R² and R⁴ are each independently for each occurrence selected from H and C₁₋₈alkyl;

R⁵ represents 0-4 substituents on the ring to which it is attached;

W is selected from

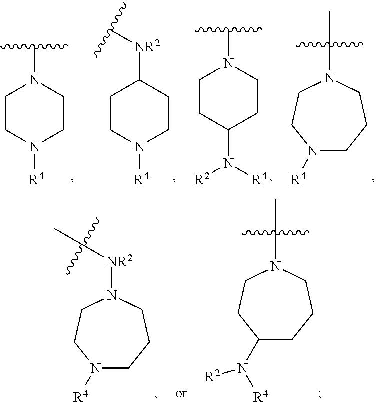

and

Q is selected from O and NR².

In certain embodiments, R⁵ represents from 1-4 substituents on the ring to which it is attached, such as halogen or nitro. In certain embodiments, R⁵ represents one substituent, such as halogen or nitro, especially chloro, situated para to the carbonyl of the quinazolinone ring. In other embodiments, R⁵ represents no substituents on the ring (i.e., all substituents are hydrogen atoms).

In preferred embodiments of formula IV, Ar is a substituted phenyl. In certain embodiments, Ar is mono-substituted wherein the substituent is halogen, lower alkoxy, or lower alkyl. In certain embodiments, Ar has a substituent at the ortho position wherein the substituent is halogen, lower alkoxy, or lower alkyl. In certain embodiments, Ar is 2,6-disubstituted such that one substituent is halogen, lower alkoxy, or lower alkyl and the second substituent is halogen, lower alkoxy, or lower alkyl.

In certain embodiments, the compounds of formula IV do not include those wherein the substituent on Ar is ethoxy at a position ortho to the bond to the nitrogen of the quinazolinone ring. In further embodiments, the compounds of formula IV do not include those wherein Ar does not have a lower alkoxy or lower alkyl substituent ortho to the bond to the nitrogen of the quinazolinone ring.

In certain embodiments of the compounds of formula IV, Ar has at least one halogen substituent. In certain embodiments, Ar has a halogen substituent in the ortho position. In preferred embodiments, the compounds of formula IV include those wherein Ar is a 2,6-disubstituted phenyl ring wherein the substituents are halogen atoms.

Exemplary compounds of formula IV include:

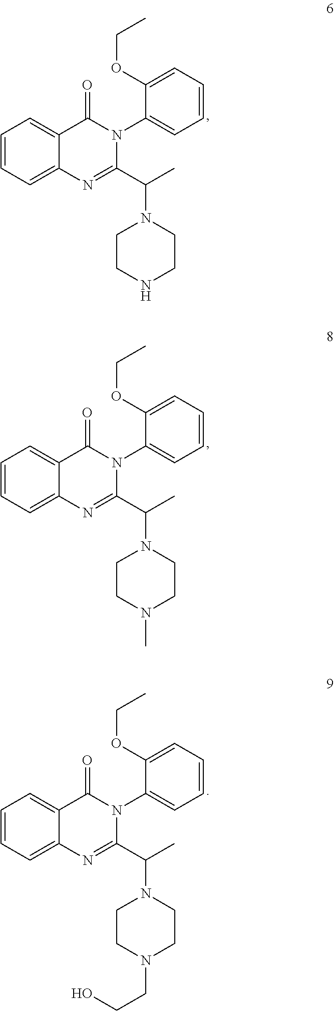 [SMILES: CCOc1ccccc1-n1c(C(C)N2CCNCC2)nc2ccccc2c1=O ; CCOc1ccccc1-n1c(C(C)N2CCN(C)CC2)nc2ccccc2c1=O ; CCOc1ccccc1-n1c(C(C)N2CCN(CCO)CC2)nc2ccccc2c1=O]

Additional compounds suitable for use in the invention are represented by the general formula V:

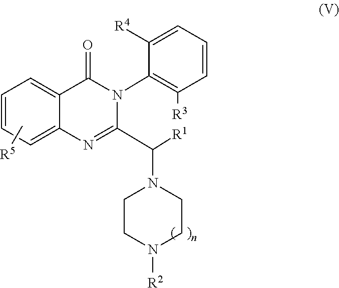

wherein:

R¹ is selected from H and C₁₋₈alkyl;

R² is selected from H and C₁₋₈alkyl;

R³ is selected from halogen, C₁₋₈alkoxy and C₁₋₈alkyl;

R⁴ is selected from H, halogen, C₁₋₈alkoxy and C₁₋₈alkyl;

R⁵ is selected from H, halogen and nitro; and

n is 1 or 2.

Exemplary compounds of formula V include:

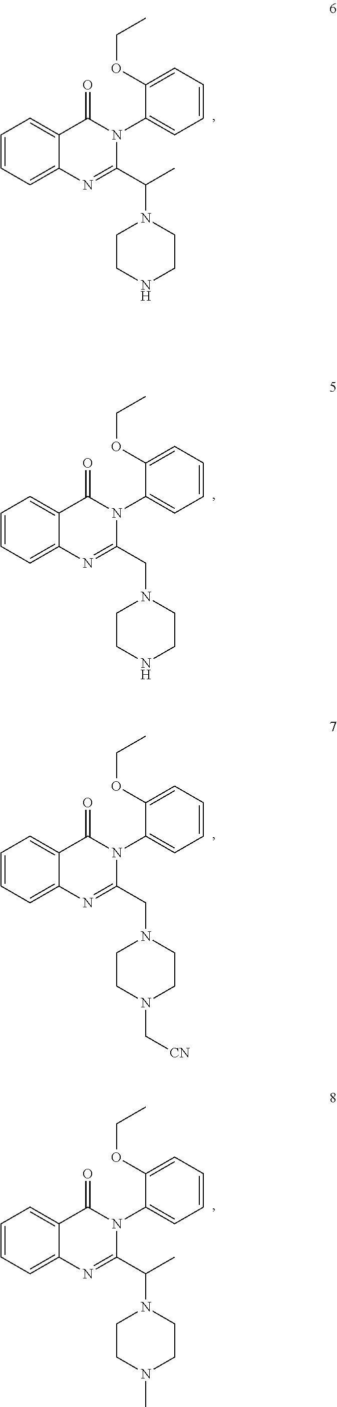 [SMILES: CCOc1ccccc1-n1c(C(C)N2CCNCC2)nc2ccccc2c1=O ; CCOc1ccccc1-n1c(CN2CCNCC2)nc2ccccc2c1=O ; CCOc1ccccc1-n1c(CN2CCN(CC#N)CC2)nc2ccccc2c1=O ; CCOc1ccccc1-n1c(C(C)N2CCN(C)CC2)nc2ccccc2c1=O]

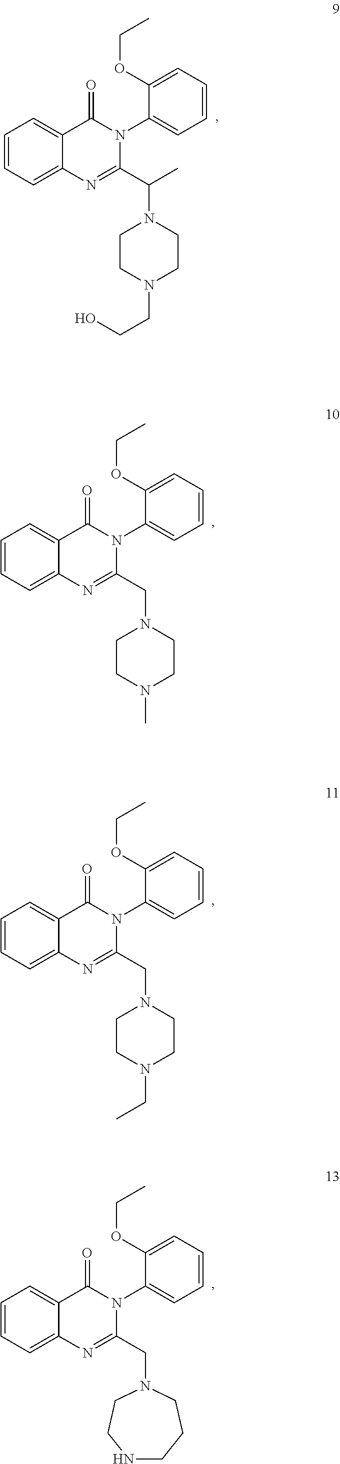 [SMILES: CCOc1ccccc1-n1c(C(C)N2CCN(CCO)CC2)nc2ccccc2c1=O ; CCOc1ccccc1-n1c(CN2CCN(C)CC2)nc2ccccc2c1=O ; CCOc1ccccc1-n1c(CN2CCN(CC)CC2)nc2ccccc2c1=O ; CCOc1ccccc1-n1c(CN2CCCNCC2)nc2ccccc2c1=O]

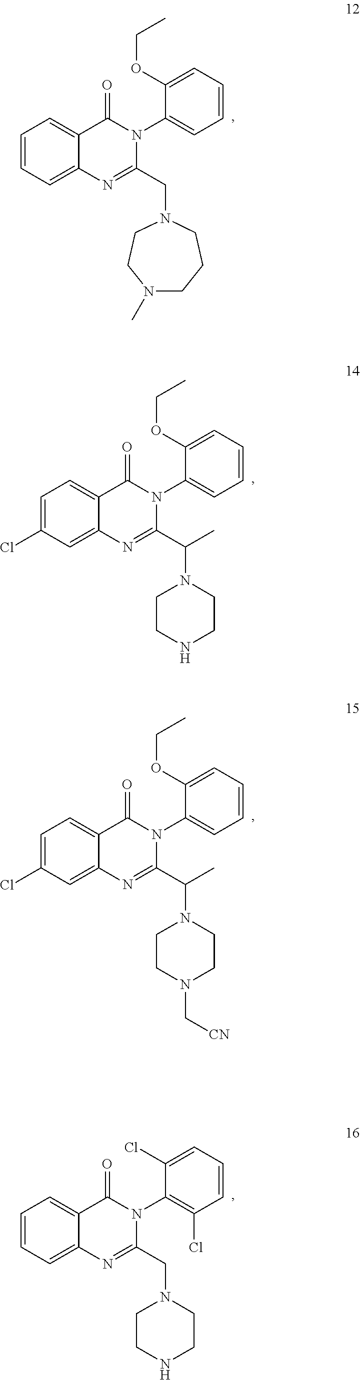 [SMILES: CCOc1ccccc1-n1c(CN2CCCN(C)CC2)nc2ccccc2c1=O ; CCOc1ccccc1-n1c(C(C)N2CCNCC2)nc2cc(Cl)ccc2c1=O ; CCOc1ccccc1-n1c(C(C)N2CCN(CC#N)CC2)nc2cc(Cl)ccc2c1=O ; O=c1c2ccccc2nc(CN2CCNCC2)n1-c1c(Cl)cccc1Cl]

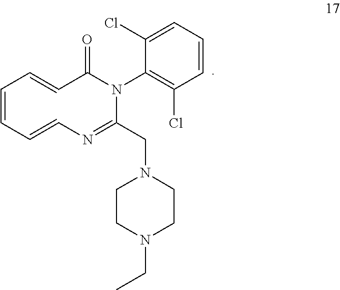 [SMILES: CCN1CCN(Cc2nccccccc(=O)n2-c2c(Cl)cccc2Cl)CC1]

Group II

One group of compounds suitable for use in the invention is represented by general formula VI:

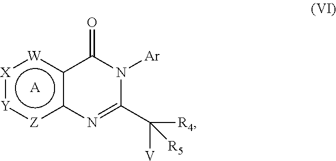

or a pharmaceutically acceptable salt thereof, where:

Ring A is optionally substituted;

W is absent or is selected from C, N, S and O;

X, Y and Z are selected from C, N, S and O, where at least one of X, Y and Z is N if W is C;

Ar is an optionally substituted phenyl group;

R₄ and R₅ are independently selected from —H, substituted or unsubstituted alkyl, substituted or unsubstituted alkenyl, substituted or unsubstituted alkynyl, substituted or unsubstituted non-aromatic heterocyclic and substituted or unsubstituted aryl, where alkyl, alkenyl and alkynyl are optionally interrupted by NR, O or S(O)ₙ; or R₄ and R₅ taken together form a 3- to 8-membered carbocyclic or heterocyclic group;

V is —NH-L-A-Q or

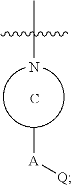

Ring C is a substituted or unsubstituted heterocyclic aromatic or non-aromatic ring;

A is NR or O; or A is a covalent bond;

L is a substituted or unsubstituted hydrocarbyl group optionally interrupted by one or more heteroatoms selected from N, O and S;

Q is selected from —R, —C(O)R′, —C(O)N(R)₂, —C(O)OR′ and —S(O)₂R′;

each R is independently —H, substituted or unsubstituted alkyl, substituted or unsubstituted alkenyl, substituted or unsubstituted alkynyl, substituted or unsubstituted aryl or substituted or unsubstituted non-aromatic heterocyclic;

each R′ is independently a substituted or unsubstituted alkyl, substituted or unsubstituted alkenyl, substituted or unsubstituted alkynyl group, substituted or unsubstituted non-aromatic heterocyclic or substituted or unsubstituted aryl group; and

each n is independently 0, 1 or 2.

In certain embodiments, W is selected from C, N, S and O.

When W is C, N, S, or O, Y or Z is typically N. In a group of compounds of the invention, W and Y are C, Z is N and X is C or N. In another group of compounds of the invention, W, X and Z are C and Y is N.

In certain embodiments, W is absent. When W is absent, at least one of X, Y and Z is N, O or S. For example, W is absent, one of X, Y and Z is S and the others are C. In a particular example, W is absent, X and Y are each C and Z is S.

For certain compounds of the invention having the values of W, X, Y and Z described above, V is

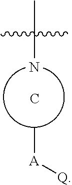

Suitable examples of V encompassed by the above structure include

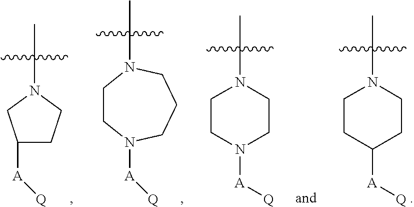

When V is represented by one of these structures, A is typically a covalent bond or NR. Particularly suitable examples of V are

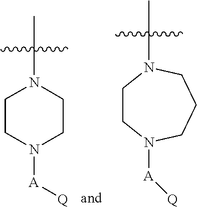

where A is a covalent bond; and

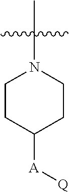

where A is NR.

In certain embodiments, A is a covalent bond and Q is —R. When Q is —R, Q is typically —H or a substituted or unsubstituted alkyl group (e.g., methyl, ethyl). In certain such embodiments, V is

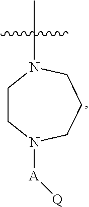

A is a covalent bond and Q is —H or methyl, particularly methyl.

In certain embodiments, the substituent -Q in compounds of the invention, particularly compounds where V is as represented above, is an acyl group. Acyl groups typically are represented by —C(O)R′, where R′ is as defined above. In certain embodiments, R′ in —C(O)R′ is a substituted or unsubstituted aryl or aryloxyalkyl group, particularly a substituted or unsubstituted phenyl or phenyloxyalkyl group such as a substituted or unsubstituted phenyloxymethyl group. Suitable substituents for the phenyl group include C₁₋₆alkyl, CF₃, hydroxyl, C₁₋₄alkoxy, aryl, aryloxy, halogen, —N(R)₂, nitro, carboxylic acid, carboxylic ester, and sulfonyl. Suitable substituents for the phenyloxymethyl group include halogens, particularly chlorine. Chlorine, when present, is preferably at the 4-position of the phenyl ring, to produce a -Q group as shown below:

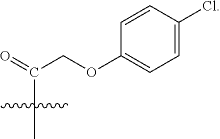 [SMILES: *C(=O)COc1ccc(Cl)cc1]

In compounds where V is represented by —NH-L-A-Q, L is typically a substituted or unsubstituted alkylene or poly(alkylene glycol) (e.g., poly(ethylene glycol), polypropylene glycol). Examples of suitable alkylene are represented by —(CH₂)ⱼ—, where j is an integer from 1 to 6, such as 2 to 4. Poly(alkylene glycols) are generally 2- or 3-mers.

R₄ and R₅ are typically independently —H or a substituted or unsubstituted alkyl group (e.g., alkyl, alkoxyalkyl, mono- or dialkylaminoalkyl, aralkyl), particularly when V (including A and Q), W, X, Y and Z have the values described above. More typically, R₄ and R₅ are independently —H or a substituted or unsubstituted C₁-C₄ alkyl group, particularly where one is —H and the other is the C₁-C₄ alkyl group.

In certain embodiments, Ring A is substituted with 1-4 substituents, such as halogen or nitro. In certain embodiments, Ring A is substituted with one substituent, such as halogen or nitro, especially chloro, situated para to the carbonyl of the quinazolinone ring. In other embodiments, there are no substituents on Ring B (i.e., all substituents are hydrogen atoms).

In preferred embodiments of the present invention, Ar is a substituted phenyl. In certain embodiments, Ar is mono-substituted wherein the substituent is halogen, lower alkoxy, or lower alkyl. In certain embodiments, Ar has a substituent at the ortho position wherein the substituent is halogen, lower alkoxy, or lower alkyl. In certain embodiments, Ar is 2,6-disubstituted such that one substituent is halogen, lower alkoxy, or lower alkyl and the second substituent is halogen, lower alkoxy, or lower alkyl.

In certain embodiments, Ar has at least one halogen substituent. In certain embodiments, Ar has a halogen substituent in the ortho position. In preferred embodiments, Ar is a 2,6-disubstituted phenyl ring wherein the substituents are halogen atoms.

The present invention also provides compounds represented by Structural Formula (VII), where the compounds are suitable for use in the methods and compositions disclosed herein:

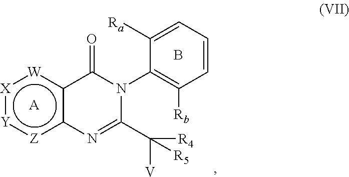

or a pharmaceutically acceptable salt thereof, where:

Rings A and B are optionally further substituted;

W is absent or is selected from C, N, S and O;

X, Y and Z are selected from C, N, S and O, where at least one of X, Y and Z is N if W is C;

Rₐ is a halogen, substituted or unsubstituted alkyl, substituted or unsubstituted alkenyl, substituted or unsubstituted alkynyl, substituted or unsubstituted aryl-O—, substituted or unsubstituted alkyl-O—, substituted or unsubstituted alkenyl-O— or substituted or unsubstituted alkynyl-O—, where alkyl, alkenyl and alkynyl are optionally interrupted by NR, O or S(O)ₙ;

R_{b} is H, halogen, C₁₋₈alkoxy, C₁₋₈alkyl, C₂₋₈alkynyl, —CF3, —OCF3, —NO₂ or —CN; typically H, halogen, C₁₋₈alkoxy or C₁₋₈alkyl;

R₄ and R₅ are independently selected from-H, substituted or unsubstituted alkyl, substituted or unsubstituted alkenyl, substituted or unsubstituted alkynyl, substituted or unsubstituted non-aromatic heterocyclic and substituted or unsubstituted aryl, where alkyl, alkenyl and alkynyl are optionally interrupted by NR, O or S(O)ₙ; or R₄ and R₅ taken together form a 3- to 8-membered carbocyclic or heterocyclic group;

V is —NH-L-A-Q or

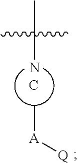

Ring C is a substituted or unsubstituted heterocyclic aromatic or non-aromatic ring;

A is NR or O; or A is a covalent bond;

L is a substituted or unsubstituted hydrocarbyl group optionally interrupted by one or more heteroatoms selected from N, O and S;

Q is selected from —R, —C(O)R′, —C(O)N(R)₂, —C(O)OR′ and —S(O)₂R′;

each R is independently —H, substituted or unsubstituted alkyl, substituted or unsubstituted alkenyl, substituted or unsubstituted alkynyl, substituted or unsubstituted aryl or substituted or unsubstituted non-aromatic heterocyclic;

each R′ is independently a substituted or unsubstituted alkyl, substituted or unsubstituted alkenyl, substituted or unsubstituted alkynyl group, substituted or unsubstituted non-aromatic heterocyclic or substituted or unsubstituted aryl group; and

each n is independently 0, 1 or 2.

In certain embodiments, W is selected from C, N, S and O.

In certain embodiments, when W is C, N, S, or O, Z is N. In a group of compounds of the invention, W and Y are C, Z is N and X is C or N, thereby resulting in compounds represented by the following structural formulas:

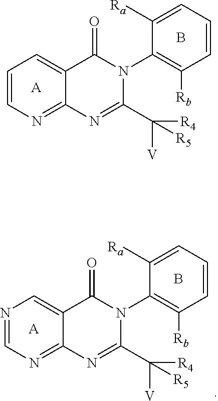

In certain embodiments, when W is C, N, S, or O, Y is N. In a group of compounds of the invention, W, X and Z are each C.

In certain embodiments, W is absent. When W is absent, at least one of X, Y and Z is N, O or S. For example, W is absent, one of X, Y and Z is S and the others are C. In a particular example, W is absent, X and Y are each C and Z is S.

For certain compounds of the invention having the values of W, X, Y and Z described above, V is

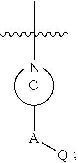

Suitable examples of V encompassed by the above structure include

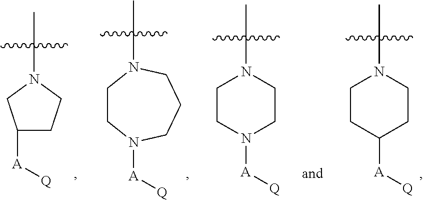

When V is represented by one of these structures, A is typically a covalent bond or NR. Particularly suitable examples of V are

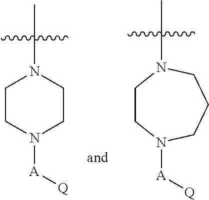

where A is a covalent bond; and

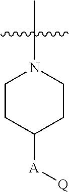

where A is NR.

In certain embodiments, A is a covalent bond and Q is —R. When Q is —R, Q is typically —H or a substituted or unsubstituted alkyl group (e.g., methyl, ethyl). In certain such embodiments, V is

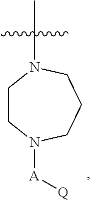

A is a covalent bond and Q is —H or methyl, particularly methyl.

In certain embodiments, the substituent -Q in compounds of the invention, particularly compounds where V is as represented above, is an acyl group. Acyl groups typically are represented by —C(O)R′, where R′ is as defined above. In certain embodiments, R′ in —C(O)R′ is a substituted or unsubstituted aryl or aryloxyalkyl group, particularly a substituted or unsubstituted phenyl or phenyloxyalkyl group such as a substituted or unsubstituted phenyloxymethyl group. Suitable substituents for the phenyl group include C₁₋₆alkyl, CF₃, hydroxyl, C₁₋₄alkoxy, aryl, aryloxy, halogen, —N(R)₂, nitro, carboxylic acid, carboxylic ester, and sulfonyl. Suitable substituents for the phenyloxymethyl group include halogens, particularly chlorine. Chlorine, when present, is preferably at the 4-position of the phenyl ring, to produce a -Q group as shown below:

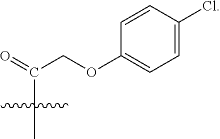 [SMILES: *C(=O)COc1ccc(Cl)cc1]

In compounds where V is represented by —NH-L-A-Q, L is typically a substituted or unsubstituted alkylene or poly(alkylene glycol) (e.g., poly(ethylene glycol), polypropylene glycol). Examples of suitable alkylene are represented by —(CH₂)ⱼ—, where j is an integer from 1 to 6, such as 2 to 4. Poly(alkylene glycols) are generally 2- or 3-mers.

R₄ and R₅ are typically independently —H or a substituted or unsubstituted alkyl group (e.g., alkyl, alkoxyalkyl, mono- or dialkylaminoalkyl, aralkyl), particularly when V (including A and Q), W, X, Y and Z have the values described above. More typically, R₄ and R₅ are independently —H or a substituted or unsubstituted C₁-C₄ alkyl group, particularly where one is —H and the other is the C₁-C₄ alkyl group.

Rₐ is typically a halogen or a substituted or unsubstituted alkyl-O— group, particularly where the alkyl portion is an unsubstituted C₁-C₄ alkyl group (e.g., methyl, ethyl, n-propyl, i-propyl, n-butyl, s-butyl, t-butyl). In one example, R₁ is typically a substituted or unsubstituted alkyl-O— group when R₄, R₅, V, W, X, Y and Z have the values described above.

R_{b} is typically —H or a halogen. In certain embodiments, R₁ is a substituted or unsubstituted alkyl-O— group and R_{b} is —H.

Although Rings A and B are typically not further substituted in compounds of the invention (i.e., no substituents are present other than those specifically shown in the Structural Formula (VI) or (VII)), Rings A and B are substituted in certain embodiments. Suitable substituents include halogen, nitro, substituted or unsubstituted alkyl, substituted or unsubstituted aryl, substituted or unsubstituted non-aromatic heterocyclic, —CN, —COOR′—CON(R)₂, —SO₂N(R)₂, —OH and —OR′, particularly —CF₃, —OCF₃, nitro and halogen. In certain embodiments, when Ring A includes two or more nitrogen atoms, one of the nitrogen atoms advantageously is substituted with a substituted or unsubstituted alkyl or aryl, typically unsubstituted. Exemplary substituents for the nitrogen atom include methyl, ethyl, n-propyl, i-propyl and phenyl.

Particularly suitable compounds of the invention have one or more of the following features: (1) V is 4-methylhomopiperazinyl, 4-ethylhomopiperazinyl, 4-(4-chlorophenoxyacetyl)piperazinyl or 4-piperazinyl, preferably 4-methylhomopiperazinyl; (2) Y or Z is N, preferably Y is N; (3) X is C or N; (4) W and Y or W and Z are C, preferably W and Z are C; (5) R₄ is —H or an unsubstituted alkyl group, preferably —H or methyl; (6) R₅ is —H or unsubstituted alkyl (e.g., methyl), preferably —H; (7) Rₐ is ethoxy and R_{b} is H or Rₐ and R_{b} are each halogen (e.g., C1); and (8) Rings A and B are not further substituted. Examples of such suitable compounds have feature (1); features (1) and (2); features (1)-(3); features (1)-(4); features (1)-(5); features (1)-(6); features (1)-(7); or features (1)-(8).

Other particularly suitable compounds of the invention have one or more of the following features: (1) V is 4-methylhomopiperazinyl, 4-ethylhomopiperazinyl, 4-(4-chlorophenoxyacetyl)piperazinyl or 4-piperazinyl, preferably 4-methylhomopiperazinyl or 4-ethylhomopiperazinyl; (2) W is absent; (3) Z is S, O or N, preferably S; (4) X and Y are C; (5) R₄ is —H or an unsubstituted alkyl group, preferably methyl; (6) R₅ is —H or unsubstituted alkyl (e.g., methyl), preferably —H; (7) Rₐ is ethoxy and R_{b} is H or Rₐ and R_{b} are each halogen (e.g., C1); and (8) Rings A and B are not further substituted. Examples of such suitable compounds have feature (1); features (1) and (2); features (1)-(3); features (1)-(4); features (1)-(5); features (1)-(6); features (1)-(7); or features (1)-(8).

The present invention also provides compounds represented by Structural Formula (VIII), where the compounds are suitable for use in the methods and compositions disclosed herein:

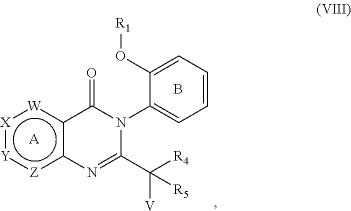

or a pharmaceutically acceptable salt thereof, where:

Rings A and B are optionally further substituted;

W is absent or is selected from C, N, S and O;

X, Y and Z are selected from C, N, S and O, where at least one of X, Y and Z is N if W is C;

R₁ is a substituted or unsubstituted alkyl, substituted or unsubstituted alkenyl or substituted or unsubstituted alkynyl group, each of which is optionally interrupted by NR, O or S(O)ₙ;

R₄ and R₅ are independently selected from —H, substituted or unsubstituted alkyl, substituted or unsubstituted alkenyl, substituted or unsubstituted alkynyl, substituted or unsubstituted non-aromatic heterocyclic and substituted or unsubstituted aryl, where alkyl, alkenyl and alkynyl are optionally interrupted by NR, O or S(O)ₙ; or R₄ and R₅ taken together form a 3- to 8-membered carbocyclic or heterocyclic group;

V is —NH-L-A-Q or

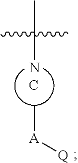

Ring C is a substituted or unsubstituted heterocyclic aromatic or non-aromatic ring;

A is NR or O; or A is a covalent bond;

L is a substituted or unsubstituted hydrocarbyl group optionally interrupted by one or more heteroatoms selected from N, O and S;

Q is selected from —R, —C(O)R′, —C(O)N(R)₂, —C(O)OR′ or —S(O)₂R′;

each R is independently —H, substituted or unsubstituted alkyl, substituted or unsubstituted alkenyl, substituted or unsubstituted alkynyl, substituted or unsubstituted aryl or substituted or unsubstituted non-aromatic heterocyclic;

each R′ is independently a substituted or unsubstituted alkyl, substituted or unsubstituted alkenyl, substituted or unsubstituted alkynyl group, substituted or unsubstituted non-aromatic heterocyclic or substituted or unsubstituted aryl group; and

each n is independently 0, 1 or 2.

In certain embodiments, W is selected from C, N, S and O.

In certain embodiments, when W is C, N, S, or O, Z is N. In a group of compounds of the invention, W and Y are C, Z is N and X is C or N, thereby resulting in compounds represented by the following structural formulas:

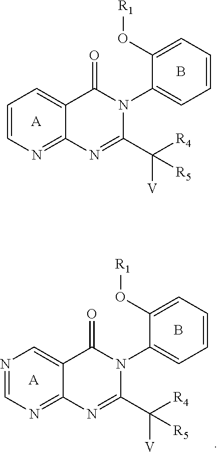

In certain embodiments, when W is C, N, S, or O, Y is N. In a group of compounds of the invention, W, X and Z are each C.

For certain compounds of the invention having the values of W, X, Y and Z described above, V is

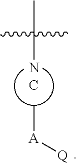

Suitable examples of V encompassed by the above structure include

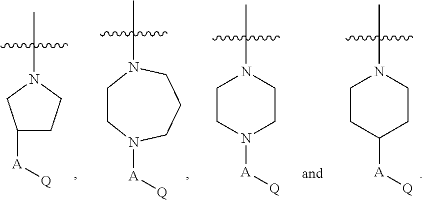

When V is represented by one of these structures, A is typically a covalent bond or NR. Particularly suitable examples of V are

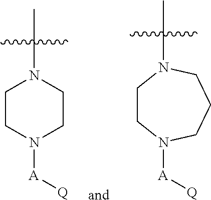

where A is a covalent bond; and

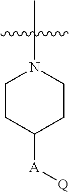

where A is NR.

In certain embodiments, A is a covalent bond and Q is —R. When Q is —R, Q is typically —H or a substituted or unsubstituted alkyl group (e.g., methyl, ethyl). In certain such embodiments, V is

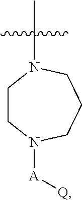

A is a covalent bond and Q is —H or methyl, particularly methyl.

In certain embodiments, the substituent -Q in compounds of the invention, particularly compounds where V is as represented above, is an acyl group. Acyl groups typically are represented by —C(O)R′, where R′ is as defined above. In certain embodiments, R′ in —C(O)R′ is a substituted or unsubstituted aryl or aryloxyalkyl group, particularly a substituted or unsubstituted phenyl or phenyloxyalkyl group such as a substituted or unsubstituted phenyloxymethyl group. Suitable substituents for the phenyl group include C₁₋₆alkyl, CF₃, hydroxyl, C₁₋₄alkoxy, aryl, aryloxy, halogen, —N(R)2, nitro, carboxylic acid, carboxylic ester, and sulfonyl. Suitable substituents for the phenyloxymethyl group include halogens, particularly chlorine. Chlorine, when present, is preferably at the 4-position of the phenyl ring, to produce a -Q group as shown below:

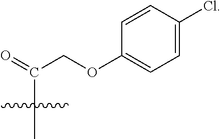 [SMILES: *C(=O)COc1ccc(Cl)cc1]

In compounds where V is represented by —NH-L-A-Q, L is typically a substituted or unsubstituted alkylene or poly(alkylene glycol) (e.g., poly(ethylene glycol), polypropylene glycol). Examples of suitable alkylene are represented by —(CH₂)ⱼ—, where j is an integer from 1 to 6, such as 2 to 4. Poly(alkylene glycols) are generally 2- or 3-mers.

R₄ and R₅ are typically independently —H or a substituted or unsubstituted alkyl group (e.g., alkyl, alkoxyalkyl, mono- or dialkylaminoalkyl, aralkyl), particularly when V (including A and Q), W, X, Y and Z have the values described above. More typically, R₄ and R₅ are independently —H or a substituted or unsubstituted C₁-C₄ alkyl group, particularly where one is —H and the other is the C₁-C₄ alkyl group.

R₁ is typically a substituted or unsubstituted alkyl group, particularly an unsubstituted C₁-C₄ alkyl group (e.g., methyl, ethyl, n-propyl, i-propyl, n-butyl, s-butyl, t-butyl). In one example, R₁ is typically a substituted or unsubstituted alkyl group when R₄, R₅, V, W, X, Y and Z have the values described above.

Although Rings A and B are typically not further substituted in compounds of the invention (i.e., no substituents are present other than those specifically shown in the Structural Formula (VIII)), Rings A and Bare substituted in certain embodiments. Suitable substituents include halogen, substituted or unsubstituted alkyl, substituted or unsubstituted aryl, substituted or unsubstituted non-aromatic heterocyclic, —CN, —COOR′, —CON(R)₂, —SO₂N(R)₂, —OH and —OR′.

Particularly suitable compounds of the invention have one or more of the following features: (1) V is V is 4-methylhomopiperazinyl, 4-ethylhomopiperazinyl, 4-(4-chlorophenoxyacetyl)piperazinyl or 4-piperazinyl, preferably 4-methylhomopiperazinyl or 4-ethylhomopiperazinyl; (2) Y or Z is N, preferably Y is N; (3) X is C or N; (4) W and Y or W and Z are C, preferably W and Z are C; (5) R₄ is —H or an unsubstituted alkyl group, preferably —H or methyl; (6) R₅ is —H or unsubstituted alkyl (e.g., methyl), preferably —H; (7) R₁ is an unsubstituted alkyl group, preferably ethyl; and (8) Rings A and B are not further substituted. Examples of such suitable compounds have feature (1); features (1) and (2); features (1)-(3); features (1)-(4); features (1)-(5); features (1)-(6); features (1)-(7); or features (1)-(8).

Exemplary compounds having all 8 features include:

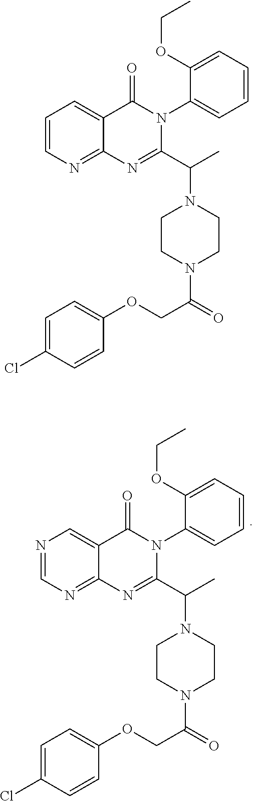 [SMILES: CCOc1ccccc1-n1c(C(C)N2CCN(C(=O)COc3ccc(Cl)cc3)CC2)nc2ncccc2c1=O ; CCOc1ccccc1-n1c(C(C)N2CCN(C(=O)COc3ccc(Cl)cc3)CC2)nc2ncncc2c1=O]

Additional exemplary compounds are provided in the examples.

In certain embodiments, W in Structural Formula (VIII) is absent, such that the encompassed compounds are represented by Structural Formula (IX):

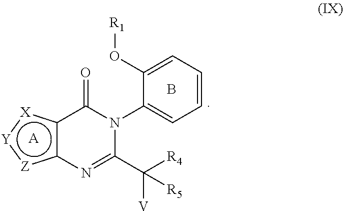

Typically, at least one of X, Y and Z is N or S. In one exemplary embodiment, Z is N, such as when X is C and Y is N. In another exemplary embodiment, Z is S, such as when X and Y are each C.

The typical values of R₁, R₄, R₅ and V (including A and Q) are identical to those discussed for compounds of Structural Formula (VI) where W is C, N, O or S.

Although Rings A and B are often unsubstituted in compounds represented by Structural Formula (IX), substitution is suitable in certain embodiments. In certain embodiments, when Ring A includes two or more nitrogen atoms, one of the nitrogen atoms is substituted with a substituted or unsubstituted alkyl or aryl, typically unsubstituted. Exemplary substituents for the nitrogen atom include methyl, ethyl, n-propyl, i-propyl and phenyl.

Particularly suitable compounds of the invention have one or more of the following features: (1) V is V is 4-methylhomopiperazinyl, 4-ethylhomopiperazinyl, 4-(4-chlorophenoxyacetyl)piperazinyl or 4-piperazinyl, preferably 4-methylhomopiperazinyl or 4-ethylhomopiperazinyl; (2) Z is N or S; (3) X is C; (4) Y is C or N; (5) R₄ is —H or an unsubstituted alkyl group, preferably —H or methyl; (6) R₅ is —H or unsubstituted alkyl (e.g., methyl), preferably —H; (7) R₁ is an unsubstituted alkyl group, preferably methyl; and (8) Rings A and B are not further substituted or when Ring A includes two or more nitrogen atoms, one of the nitrogen atoms is substituted with an unsubstituted alkyl or aryl. Examples of such suitable compounds have feature (1); features (1) and (2); features (1)-(3); features (1)-(4); features (1)-(5); features (1)-(6); features (1)-(7); or features (1)-(8).

Exemplary compounds having all 8 features include:

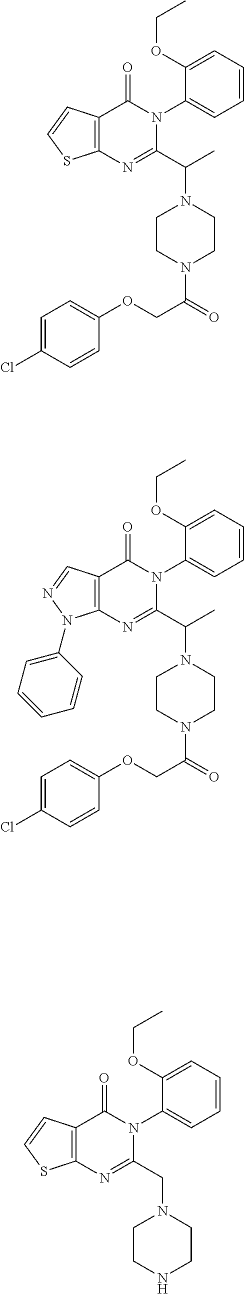 [SMILES: CCOc1ccccc1-n1c(C(C)N2CCN(C(=O)COc3ccc(Cl)cc3)CC2)nc2sccc2c1=O ; CCOc1ccccc1-n1c(C(C)N2CCN(C(=O)COc3ccc(Cl)cc3)CC2)nc2c(cnn2-c2ccccc2)c1=O ; CCOc1ccccc1-n1c(CN2CCNCC2)nc2sccc2c1=O]

Group III

The present invention also provides compounds represented by Structural Formula (X), where the compounds are suitable for use in the methods and compositions disclosed herein:

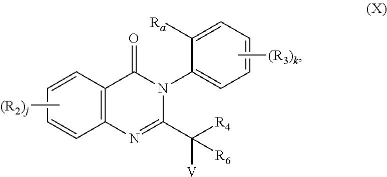

or a pharmaceutically acceptable salt thereof, where:

Rₐ is a halogen, substituted or unsubstituted alkyl, substituted or unsubstituted alkenyl, substituted or unsubstituted alkynyl, substituted or unsubstituted aryl-O—, substituted or unsubstituted alkyl-O—, substituted or unsubstituted alkenyl-O— or substituted or unsubstituted alkynyl-O—, where alkyl, alkenyl and alkynyl are optionally interrupted by NR, O or S(O)ₙ;

each R₂ is independently selected from halogen, substituted or unsubstituted alkyl, substituted or unsubstituted aryl, substituted or unsubstituted non-aromatic heterocyclic, —CN, —COOR′, —CON(R)₂, —NRC(O)R, —SO₂N(R)₂, —N(R)₂, —NO₂, —OH and —OR′;

each R₃ is independently selected from halogen, substituted or unsubstituted alkyl, substituted or unsubstituted aryl, substituted or unsubstituted non-aromatic heterocyclic, —CN, —COOR′, —CON(R)₂, —NRC(O)R, —SO₂N(R)₂, —N(R)₂, —NO₂, —OH and —OR′;

R₄ and R₅ are independently selected from —H, substituted or unsubstituted alkyl, substituted or unsubstituted alkenyl, substituted or unsubstituted alkynyl, substituted or unsubstituted non-aromatic heterocyclic and substituted or unsubstituted aryl, where alkyl, alkenyl and alkynyl are optionally interrupted by NR, O or S(O)ₙ; or R₄ and R₅ taken together form a carbocyclic or heterocyclic group;

V is —NH-L-A-Q or

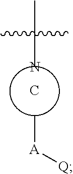

Ring C is a substituted or unsubstituted heterocyclic aromatic or non-aromatic ring;

A is NR or O; or A is a covalent bond;

L is a substituted or unsubstituted hydrocarbyl group optionally interrupted by one or more heteroatoms selected from N, O and S;

Q is selected from —R, —C(O)R′—C(O)N(R)₂, —C(O)OR′ and —S(O)₂R′;

each R is independently —H, substituted or unsubstituted alkyl, substituted or unsubstituted alkenyl, substituted or unsubstituted alkynyl, substituted or unsubstituted aryl or substituted or unsubstituted non-aromatic heterocyclic;

each R′ is independently a substituted or unsubstituted alkyl, substituted or unsubstituted alkenyl, substituted or unsubstituted alkynyl group, substituted or unsubstituted non-aromatic heterocyclic or substituted or unsubstituted aryl group;

j is an integer from 0 to 4;

k is an integer from 0 to 4, provided that at least one of j and k is an integer from 1 to 4; and

each n is independently 0, 1 or 2.

For certain compounds of the invention, V is

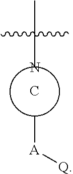

Suitable examples of V encompassed by the above structure include

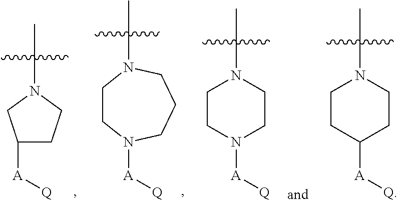

When V is represented by one of these structures, A is typically a covalent bond or NR. Particularly suitable examples of V are

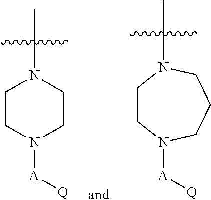

where A is a covalent bond; and

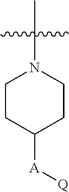

where A is NR.

In certain embodiments, A is a covalent bond and Q is —R. When Q is —R, Q is typically —H or a substituted or unsubstituted alkyl group (e.g., methyl, ethyl). In certain such embodiments, V is

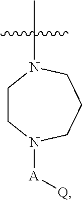

A is a covalent bond and Q is —H, methyl or ethyl, particularly methyl.

In certain embodiments, the substituent -Q in compounds of the invention, particularly compounds where V is as represented above, is an acyl group. Acyl groups typically are represented by —C(O)R′, where R′ is as defined above. In certain embodiments, R′ in —C(O)R′ is a substituted or unsubstituted aryl or aryloxyalkyl group, particularly a substituted or unsubstituted phenyl or phenyloxyalkyl group such as a substituted or unsubstituted phenyloxymethyl group. Suitable substituents for the phenyl group include C₁₋₆alkyl, CF₃, hydroxyl, C₁₋₄alkoxy, aryl, aryloxy, halogen, —N(R)₂, nitro, carboxylic acid, carboxylic ester, and sulfonyl. Suitable substituents for the phenyloxymethyl group include halogens, particularly chlorine. Chlorine, when present, is preferably at the 4-position of the phenyl ring, to produce a -Q group as shown below:

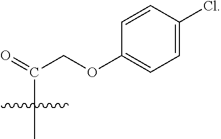 [SMILES: *C(=O)COc1ccc(Cl)cc1]

In compounds where V is represented by —NH-L-A-Q, L is typically a substituted or unsubstituted alkylene or poly(alkylene glycol) (e.g., poly(ethylene glycol), polypropylene glycol). Examples of suitable alkylene are represented by —(CH₂)ⱼ—, where j is an integer from 1 to 6, such as 2 to 4. Poly(alkylene glycols) are generally 2- or 3-mers.

R₄ and R₅ are typically independently —H or a substituted or unsubstituted alkyl group (e.g., alkyl, alkoxyalkyl, mono- or dialkylaminoalkyl, aralkyl), particularly when V (including A and Q), has the values described above. More typically, R₄ and R₅ are independently or a substituted or unsubstituted C₁-C₄ alkyl group, particularly methyl.

R₁ is typically a substituted or unsubstituted alkyl group, particularly an unsubstituted C₁-C₄ alkyl group (e.g., methyl, ethyl, n-propyl, i-propyl, n-butyl, s-butyl, t-butyl). In one example, R₁ is typically a substituted or unsubstituted alkyl group when R₄, R₅, and V have the values described above.

In certain embodiments, j is 1, 2, 3 or 4, such as when k is 0. In certain embodiments, k is 1, 2, 3 or 4, such as when j is 0. In certain embodiments, j is an integer from 1 to 4 and k is an integer from 1 to 4. For example, j is 1 and k is 1, j is 1 and k is 2, j is 1 and k is 3, j is 1 and k is 4, j is 2 and k is 1, j is 2 and k is 2, j is 2 and k is 3, j is 2 and k is 4, j is 3 and k is 1, j is 3 and k is 2, j is 3 and k is 3, j is 3 and k is 4, j is 4 and k is 1, j is 4 and k is 2, j is 4 and k is 3, or j is 4 and k is 4.

When one or more R₂ and/or R₃ substituent groups are present, they are generally independently selected from polar substituted alkyl, polar substituted alkoxy, polar substituted carbocyclic aryl, substituted or unsubstituted heteroaryl (e.g., nitrogen-containing heteroaryl such as imidazolyl, oxazolyl, thiazolyl, pyridinyl, pyrimidinyl, triazolyl) and substituted or unsubstituted non-aromatic heterocyclic (e.g., pyrrazolyl, piperadinyl, piperazinyl, morpholinyl, homopiperazinyl). Advantageously, these groups improve the water solubility of the compound. Particularly suitable polar substituents include amino, amido, guanidino, —SO₃H, —PO₃H, —OH and —COOH (including esters that hydrolyze to —COOH), including salts thereof. Other suitable substituents include nitro, halogens such as chlorine, bromine and iodine, and halogen-substituted alkyl and alkoxy groups (e.g., —CF₃, —OCF₃).

Additional suitable values for R₂ and/or R₃ include —NRC(O)R and —N(R)₂, particularly —NHC(O)R and —NHR. For —NHC(O)R and —NHR, R is typically —H or a substituted alkyl group. The substituents on such alkyl groups are advantageously groups that are able to react with another functional group to form a covalent bond, such as an amine, carboxylic acid, acid halide, halogen or the like. Preferably, R is an aminoalkyl (e.g., where the alkyl is typically C₃-C₆) when R₂ and/or R₃ is —NHC(O)R or —NHR or R is —H when R₂ and/or R₃ is —NHR. Examples of R₂ and/or R₃ include —NH₂, —NHC(O)(CH₂)₃NH₂ and —NH(CH₂)₆NH₂.

In certain embodiment, compounds of Structural Formula (X) are represented by the following particular structures:

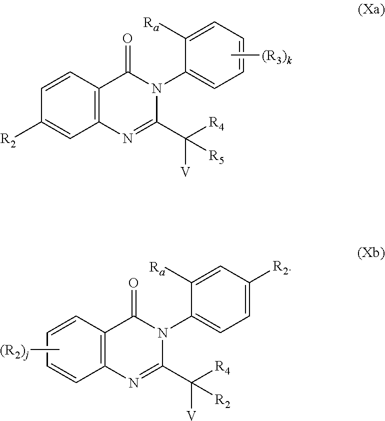

In Structural Formula (Ia), R₂ is typically —NHR (e.g., —NH₂) and Rₐ is typically alkoxy (e.g., methoxy, ethoxy). In Structural Formula (Xb), R₃ is typically a halogen or —OCF₃ and Rₐ is typically a halogen or alkoxy (e.g., methoxy, ethoxy). In certain embodiments, R₃ in Structural Formula (Ia) is present in the same location as R₃ in Structural Formula (Xb).

Particularly suitable compounds of the invention have one or more of the following features: (1) V is 4-piperazinyl, 4-homopiperazinyl, 4-methylhomopiperazinyl or 4-(4-chlorophenoxyacetyl)piperazinyl, preferably 4-methylhomopiperazinyl; (2) R₄ is —H or an unsubstituted alkyl group, preferably —H or methyl; (3) R₅ is —H or an unsubstituted alkyl group, preferably —H or methyl; (4) Rₐ is an unsubstituted alkyl-O— group, preferably ethyl-O— (i.e., ethoxy); and (5) at least one of R₂ and R₃ is a group that enhances water solubility (e.g, —NH₂), —NO₂, —OCF₃ and/or a halogen. Examples of such suitable compounds have feature (1); features (1) and (2); features (1)-(3); features (1)-(4); or features (1)-(5).

The present invention also provides compounds represented by Structural Formula (XI), where the compounds are suitable for use in the methods and compositions disclosed herein:

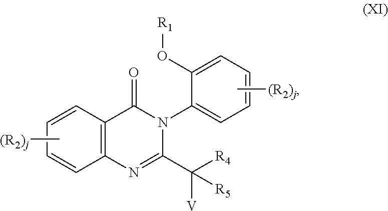

or a pharmaceutically acceptable salt thereof, where:

R₁ is a substituted or unsubstituted alkyl, substituted or unsubstituted alkenyl or substituted or unsubstituted alkynyl group, each of which is optionally interrupted by NR, O or S(O)ₙ;

each R₂ is independently selected from halogen, substituted or unsubstituted alkyl, substituted or unsubstituted aryl, substituted or unsubstituted non-aromatic heterocyclic, —CN, —COOR′, —CON(R)₂, —NRC(O)R, —SO₂N(R)₂, —N(R)₂, —NO₂, —OH and —OR′ (e.g., halogen, substituted or unsubstituted alkyl, substituted or unsubstituted aryl, substituted or unsubstituted non-aromatic heterocyclic, —CN, —COOR′, —CON(R)₂, —SO₂N(R)₂, —OH and —OR′);

each R₃ is independently selected from halogen, substituted or unsubstituted alkyl, substituted or unsubstituted aryl, substituted or unsubstituted non-aromatic heterocyclic, —CN, —COOR′, —CON(R)₂, —NRC(O)R, —SO₂N(R)₂, —N(R)₂, —NO₂, —OH and —OR′ (e.g., halogen, substituted or unsubstituted alkyl, substituted or unsubstituted aryl, substituted or unsubstituted non-aromatic heterocyclic, —CN, —COOR′, —CON(R)₂, —SO₂N(R)₂, —OH and —OR′);

R₄ and R₅ are independently selected from —H, substituted or unsubstituted alkyl, substituted or unsubstituted alkenyl, substituted or unsubstituted alkynyl, substituted or unsubstituted non-aromatic heterocyclic and substituted or unsubstituted aryl, where alkyl, alkenyl and alkynyl are optionally interrupted by NR, O or S(O)ₙ; or R₄ and R₅ taken together form a carbocyclic or heterocyclic group;

V is —NH-L-A-Q or

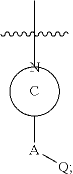

Ring C is a substituted or unsubstituted heterocyclic aromatic or non-aromatic ring;

A is NR or O; or A is a covalent bond;

L is a substituted or unsubstituted hydrocarbyl group optionally interrupted by one or more heteroatoms selected from N, O and S;

Q is selected from —R, —C(O)R′, —C(O)N(R)₂, —C(O)OR′ and —S(O)₂R′;

each R is independently —H, substituted or unsubstituted alkyl, substituted or unsubstituted alkenyl, substituted or unsubstituted alkynyl, substituted or unsubstituted aryl or substituted or unsubstituted non-aromatic heterocyclic;

each R′ is independently a substituted or unsubstituted alkyl, substituted or unsubstituted alkenyl, substituted or unsubstituted alkynyl group, substituted or unsubstituted non-aromatic heterocyclic or substituted or unsubstituted aryl group;

j is an integer from 0 to 4;

k is an integer from 0 to 4, provided that at least one of j and k is an integer from 1 to 4; and

each n is independently 0, 1 or 2.

For certain compounds of the invention, V is

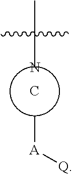

Particularly suitable examples of V are

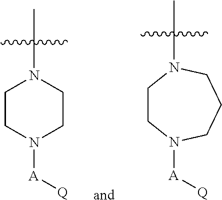

where A is a covalent bond; and

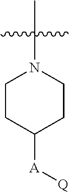

where A is NR.

In certain embodiments, A is a covalent bond and Q is —R. When Q is —R, Q is typically —H or a substituted or unsubstituted alkyl group (e.g., methyl, ethyl). In certain such embodiments, V is

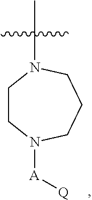

A is a covalent bond and Q is —H, methyl or ethyl, particularly methyl.

In certain embodiments, the substituent -Q in compounds of the invention, particularly compounds where V is as represented above is an acyl group. Acyl groups typically are represented by —C(O)R′, where R′ is as defined above. In certain embodiments, R′ in —C(O)R′ is a substituted or unsubstituted aryl or aryloxyalkyl group, particularly a substituted or unsubstituted phenyl or phenyloxyalkyl group such as a substituted or unsubstituted phenyloxymethyl group. Suitable substituents for the phenyl group include C₁₋₆alkyl, CF₃, hydroxyl, C₁₋₄alkoxy, aryl, aryloxy, halogen, —N(R)₂, nitro, carboxylic acid, carboxylic ester, and sulfonyl. Suitable substituents for the phenyloxymethyl group include halogens, particularly chlorine. Chlorine, when present, is preferably at the 4-position of the phenyl ring, to produce a -Q group as shown below:

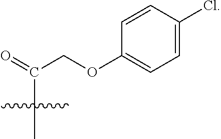 [SMILES: *C(=O)COc1ccc(Cl)cc1]

In compounds where V is represented by —NH-L-A-Q, L is typically a substituted or unsubstituted alkylene or poly(alkylene glycol) (e.g., poly(ethylene glycol), polypropylene glycol). Examples of suitable alkylene are represented by —(CH₂)ⱼ—, where j is an integer from 1 to 6, such as 2 to 4. Poly(alkylene glycols) are generally 2- or 3-mers.

R₄ and R₅ are typically independently —H or a substituted or unsubstituted alkyl group (e.g., alkyl, alkoxyalkyl, mono- or dialkylaminoalkyl, aralkyl), particularly when V (including A and Q) has the values described above. More typically, R₄ and R₅ are independently —H or a substituted or unsubstituted C₁-C₄ alkyl group, particularly methyl.

R₁ is typically a substituted or unsubstituted alkyl group, particularly an unsubstituted C₁-C₄ alkyl group (e.g., methyl, ethyl, n-propyl, i-propyl, n-butyl, s-butyl, t-butyl). In one example, R₁ is typically a substituted or unsubstituted alkyl group when R₄, R₅, and V have the values described above.

In certain embodiments, j is 1, 2, 3 or 4, such as when k is 0. In certain embodiments, k is 1, 2, 3 or 4, such as when j is 0. In certain embodiments, j is an integer from 1 to 4 and k is an integer from 1 to 4. For example, j is 1 and k is 1, j is 1 and k is 2, j is 1 and k is 3, j is 1 and k is 4, j is 2 and k is 1, j is 2 and k is 2, j is 2 and k is 3, j is 2 and k is 4, j is 3 and k is 1, j is 3 and k is 2, j is 3 and k is 3, j is 3 and k is 4, j is 4 and k is 1, j is 4 and k is 2, j is 4 and k is 3, or j is 4 and k is 4.

When one or more R₂ and/or R₃ substituent groups are present, they are generally independently selected from polar substituted alkyl, polar substituted alkoxy, polar substituted carbocyclic aryl, substituted or unsubstituted heteroaryl (e.g., nitrogen-containing heteroaryl such as imidazolyl, oxazolyl, thiazolyl, pyridinyl, pyrimidinyl, triazolyl) and substituted or unsubstituted non-aromatic heterocyclic (e.g., pyrrazolyl, piperadinyl, piperazinyl, morpholinyl, homopiperazinyl). Advantageously, these groups improve the water solubility of the compound. Particularly suitable polar substituents include amino, amido, guanidino, —SO₃H, —PO₃H, —OH and —COOH (including esters that hydrolyze to —COOH), including salts thereof. Other suitable substituents include nitro, halogens such as chlorine, bromine and iodine, and halogen-substituted alkyl and alkoxy groups (e.g., —CF₃, —OCF₃).

Additional suitable values for R₂ and/or R₃ include —NRC(O)R and —N(R)₂, particularly —NHC(O)R and —NHR. For —NHC(O)R and —NHR, R is typically —H or a substituted alkyl group. The substituents on such alkyl groups are advantageously groups that are able to react with another functional group to form a covalent bond, such as an amine, carboxylic acid, acid halide, halogen or the like. Preferably, R is an aminoalkyl (e.g., where the alkyl is typically C₃-C₆) when R₂ and/or R₃ is —NHC(O)R or —NHR or R is —H when R₂ and/or R₃ is —NHR. Examples of R₂ and/or R₃ include —NH₂, —NHC(O)(CH₂)₃NH₂ and —NH(CH₂)₆NH₂.

Particularly suitable compounds of the invention have one or more of the following features: (1) V is 4-piperazinyl, 4-homopiperazinyl, 4-methylhomopiperazinyl or 4-(4-chlorophenoxyacetyl)piperazinyl, preferably 4-methylhomopiperazinyl; (2) R₄ is an unsubstituted alkyl group, preferably methyl; (3) R₅ is —H or an unsubstituted alkyl group, preferably —H or methyl; (4) R₁ is an unsubstituted alkyl group, preferably ethyl; and (5) at least one of R₂ and R₃ is a group that enhances water solubility, —OCF₃, —NO₂ and/or a halogen. Examples of such suitable compounds have feature (1); features (1) and (2); features (1)-(3); features (1)-(4); or features (1)-(5).

Exemplary compounds include Compounds (1), (2) and (3):

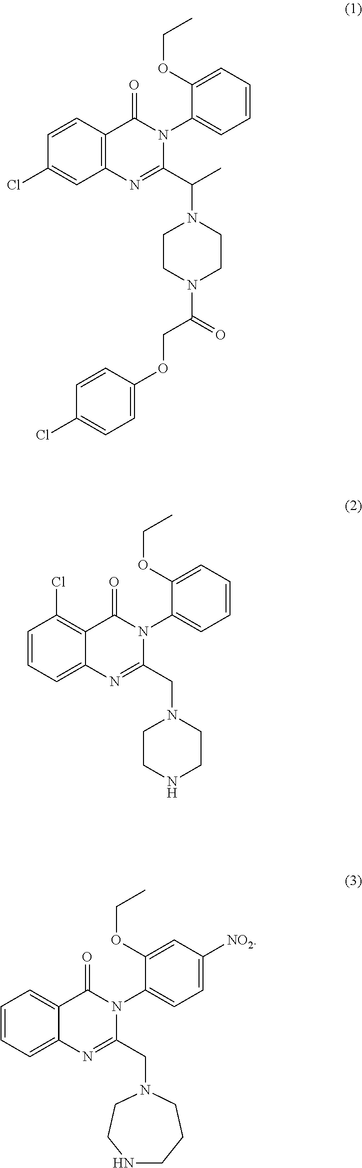 [SMILES: CCOc1ccccc1-n1c(C(C)N2CCN(C(=O)COc3ccc(Cl)cc3)CC2)nc2cc(Cl)ccc2c1=O ; CCOc1ccccc1-n1c(CN2CCNCC2)nc2cccc(Cl)c2c1=O ; CCOc1cc([N+](=O)[O-])ccc1-n1c(CN2CCCNCC2)nc2ccccc2c1=O]

Additional exemplary compounds include Compounds (4)-(9):

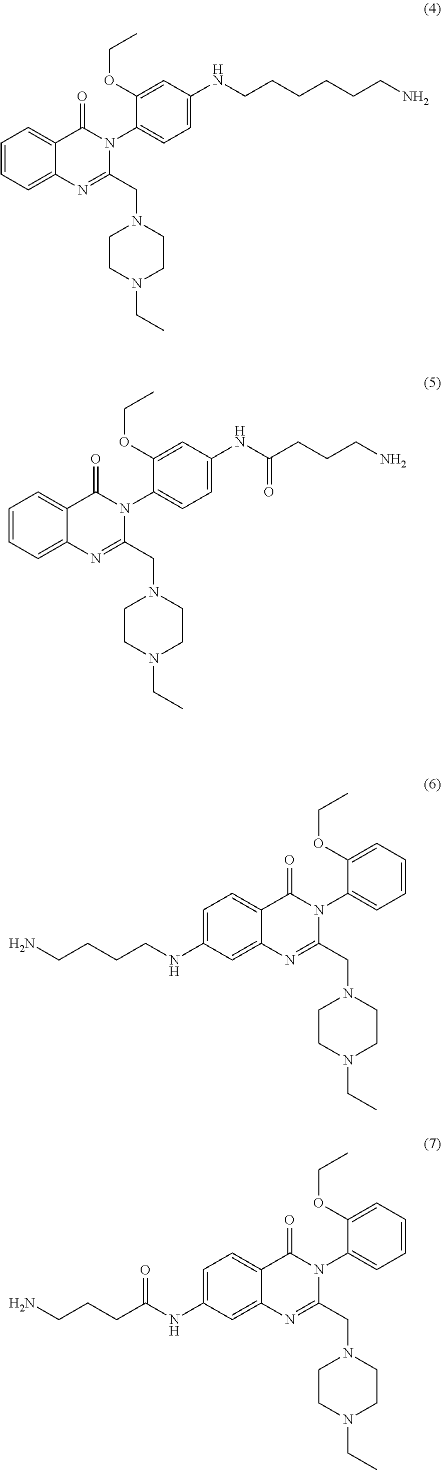 [SMILES: CCOc1cc(NCCCCCCN)ccc1-n1c(CN2CCN(CC)CC2)nc2ccccc2c1=O ; CCOc1cc(NC(=O)CCCN)ccc1-n1c(CN2CCN(CC)CC2)nc2ccccc2c1=O ; CCOc1ccccc1-n1c(CN2CCN(CC)CC2)nc2cc(NCCCCN)ccc2c1=O ; CCOc1ccccc1-n1c(CN2CCN(CC)CC2)nc2cc(NC(=O)CCCN)ccc2c1=O]

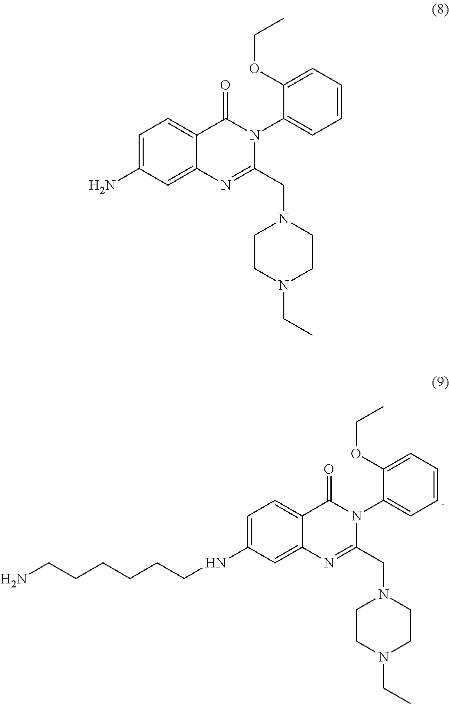 [SMILES: CCOc1ccccc1-n1c(CN2CCN(CC)CC2)nc2cc(N)ccc2c1=O ; CCOc1ccccc1-n1c(CN2CCN(CC)CC2)nc2cc(NCCCCCCN)ccc2c1=O]

Compounds included in the invention include enantiomers and diastereomers of the compounds disclosed herein. The invention also includes salts, particularly pharmaceutically acceptable salts of the compounds disclosed herein. In addition, the invention includes solvates, hydrates and polymorph crystalline forms of the compounds disclosed herein.

The compounds of the present invention that possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion (e.g., a halide such as bromide, chloride, or fluoride, particularly bromide).

Acids commonly employed to form acid addition salts are inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, p-bromophenyl-sulfonic acid, carbonic acid, succinic acid, citric acid, benzoic acid, acetic acid, and the like. Examples of such salts include the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, gamma-hydroxybutyrate, glycolate, tartrate, methanesulfonate, propanesulfonate, naphthalene-1-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, and the like.

It is contemplated that all embodiments of the invention can be combined with one or more other embodiments, even those described under different aspects of the invention.

The term “acyl” as used herein includes such moieties as can be represented by the general formula:

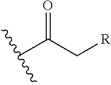

wherein suitable R groups, include, but are not limited to H, alkyl, alkoxy, aralkyl, aryloxy, aryl, heteroaryl, heteroaralkyl, heteroaryloxy, and cycloalkyl, wherein any of these groups may optionally be further appropriately substituted.

The term “hydrocarbyl” refers to substituted or unsubstituted, cyclic or acyclic, saturated or unsaturated hydrocarbon groups. When indicated, hydrocarbyl atoms can be interrupted by one or more heteroatoms such as N, O and S (i.e., the heteroatoms are not at a terminus of the group). The term “alkyl” refers to substituted or unsubstituted saturated hydrocarbon groups, including straight-chain alkyl and branched-chain alkyl groups, including haloalkyl groups such as trifluoromethyl and 2,2,2-tirfluoroethyl, etc. C₀ alkyl indicates a hydrogen where the group is in a terminal position, a bond if internal. The terms “alkenyl” and “alkynyl” refer to substituted or unsubstituted unsaturated aliphatic groups analogous possible substitution to the alkyls described above, but that contain at least one double or triple bond respectively.

The term “alkoxy” refers to an oxygen having an alkyl group attached thereto. Representative alkoxy groups include methoxy, ethoxy, propoxy, tert-butoxy and the like. An “ether” is two hydrocarbons covalently linked by an oxygen. Accordingly, the substituent of an alkyl that renders that alkyl an ether is or resembles an alkoxy.

The term “aralkyl”, as used herein, refers to an alkyl group substituted with an aryl group.

The term “carbocyclic” as used herein includes 3- to 8-membered substituted or unsubstituted single-ring saturated or unsaturated cyclic aliphatic groups in which each atom of the ring is carbon.

The term “heterocyclic” as used herein includes 3- to 8-membered, preferably 4- to 8-membered, substituted or unsubstituted single-ring cyclic groups in which the ring includes 1 to 3 heteroatoms. Examples of non-aromatic heterocyclic groups include pyrrolidine, piperadine, piperazine, tetrahydrofuran and tetrahydrothiophene.

The term “aryl” as used herein includes 5-, 6-, and 7-membered substituted or unsubstituted single-ring carbocyclic or heterocyclic aromatic groups. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls and/or heterocyclyls. Carbocyclic aryl groups include benzene, naphthalene, phenanthrene, phenol, aniline, and the like. The term “heteroaryl” includes substituted or unsubstituted aromatic 5- to 7-membered ring structures, more preferably 5- to 6-membered rings, whose ring structures include one to four heteroatoms. The term “heteroaryl” also includes polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, and/or heterocyclyls. Heteroaryl groups include, for example, pyrrole, furan, thiophene, imidazole, oxazole, thiazole, triazole, pyrazole, pyridine, pyrazine, pyridazine and pyrimidine, and the like.

The term “heteroatom” as used herein means an atom of any element other than carbon or hydrogen. Preferred heteroatoms are nitrogen, oxygen, phosphorus, and sulfur.

The terms “polycyclyl” or “polycyclic” refer to two or more rings (e.g., cycloalkyls, cycloalkenyls, cycloalkynyls, aryls, heteroaryls, and/or heterocyclyls) in which two or more carbons are common to two adjoining rings, e.g., the rings are “fused rings”. Each of the rings of the polycycle can be substituted or unsubstituted.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include, for example, a halogen, a hydroxyl, a carbonyl (such as a carboxyl, an alkoxycarbonyl, a formyl, or an acyl), a thiocarbonyl (such as a thioester, a thioacetate, or a thiofonnate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

### Methods of Treatment

In certain embodiments, the invention provides a method to treat or prevent cancer in an individual. The terms “cancer,” “tumor,” and “neoplasia” are used interchangeably herein. As used herein, a cancer (tumor or neoplasia) is characterized by one or more of the following properties: cell growth is not regulated by the normal biochemical and physical influences in the environment; anaplasia (e.g., lack of normal coordinated cell differentiation); and in some instances, metastasis. Cancer diseases include, for example, anal carcinoma, bladder carcinoma, breast carcinoma, cervix carcinoma, chronic lymphocytic leukemia, chronic myelogenous leukemia, endometrial carcinoma, fibrosarcoma, hairy cell leukemia, head and neck carcinoma, lung (small cell) carcinoma, multiple myeloma (including multiple myeloma associated with OPM-2 cells, OPM-2-like cells, MM-1S cells, MM-1S-like cells, MM.1R cells, MM-1R-like cells, KMS-18 cells, KMS-18-like cells, S6B45 cells, S6B45-like cells, MR20 cells, MR20-like cells, ARD cells and/or ARD-like cells), non-Hodgkin's lymphoma, follicular lymphoma, ovarian carcinoma, brain tumors, colorectal carcinoma, hepatocellular carcinoma, Kaposi's sarcoma, leiomyosarcoma, lung (non-small cell carcinoma), melanoma, mesenchymal chondrosarcoma, pancreatic carcinoma, prostate carcinoma, renal cell carcinoma, and soft tissue sarcoma. Other cancer diseases include leiomyosarcoma and mesenchymal chondrosarcoma. Additional cancer disorders can be found in, for example, Isselbacher et al. (1994) Harrison's Principles of Internal Medicine 1814-1877, herein incorporated by reference.

Cell types that can be treated with compounds of the invention include A-549, NCI-H1734, Calu-1, A-427, Calu-6, DLD-1, OVCAR-5, HS766T, CFPAC-1, Capan-2, HT-29, CCD841, SK-MEL-2, SU.86.86, COLO-205, AsPC-1, HUVEC, BxPC-3, and Capan-1.

In certain embodiments, compounds of the invention can be used to treat a patient suffering from a resistant cancer, particularly a resistant multiple myeloma. A resistant cancer is a cancer that is not treatable with one or more anticancer agents. Examples of anticancer agents that certain cancers, particularly multiple myeloma, are resistant to include dexamethasone, alkylators, anthracyclines (e.g., doxorubicin), lenalidomide, CC-4047, bortezomib, and multitargeted kinase inhibitors.

Typically, the cancers described above and treatable by the methods described herein exhibit deregulated Ras pathway activity. In one embodiment, the cancers described above contain a mutation in the Ras signaling pathway, resulting in elevated Ras signaling activity. For example, the mutation could be a constitutively active mutation in the Ras gene, such as Ras V12. The mutation could also be in any of the Ras-pathway related genes that could result in activation or altered activity of the pathway.

In one embodiment, the invention relates to a method of treating or preventing cancer in an individual, comprising administering to the individual a therapeutically effective amount of a compound that is selectively toxic to an engineered human tumorigenic cell, or a cancer cell of specific genotype (or specifically altered genotype). In certain embodiments, the cancer is characterized by cells comprising an activated RAS pathway. In certain further embodiments, the cancer is characterized by cells expressing SV40 small T oncoprotein, or exhibiting modulations of targets of sT and/or oncogenic RAS.

In a related embodiment, the invention contemplates the practice of the method of the invention in conjunction with other anti-tumor therapies such as conventional chemotherapy directed against solid tumors and for control of establishment of metastases. The administration of the other anti-tumor therapies can be conducted during or after chemotherapy. Such agents are typically formulated with a pharmaceutically acceptable carrier, and can be administered intravenously, orally, bucally, parenterally, by an inhalation spray, by topical application or transdermally. An agent can also be administered by local administration. Preferably, one or more additional agents administered in conjunction with an anti-cancer chemotherapeutic agent (e.g., a compound of the invention) inhibits cancer cells in an additive or synergistic manner.

A wide array of conventional compounds has been shown to have anti-tumor activities. These compounds have been used as pharmaceutical agents in chemotherapy to shrink solid tumors, prevent metastases and further growth, or decrease the number of malignant cells in leukemic or bone marrow malignancies.

Although chemotherapy has been effective in treating various types of malignancies, many anti-tumor compounds induce undesirable side effects. In many cases, when two or more different treatments are combined, the treatments may work synergistically and allow reduction of dosage of each of the treatments, thereby reducing the detrimental side effects exerted by each compound at higher dosages. In other instances, malignancies that are refractory to a treatment may respond to a combination therapy of two or more different treatments.

Therefore, compounds and pharmaceutical compositions of the present invention may be conjointly administered with a conventional anti-tumor compound. Conventional anti-tumor compounds include, merely to illustrate: aminoglutethimide, amsacrine, anastrozole, asparaginase, bcg, bevacizumab, bicalutamide, bleomycin, buserelin, busulfan, camptothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

In other embodiments, compounds and pharmaceutical compositions of the present invention may be conjointly administered with a conventional anti-tumor compound selected from: an EGF-receptor antagonist, arsenic sulfide, adriamycin, cisplatin, carboplatin, cimetidine, carminomycin, mechlorethamine hydrochloride, pentamethylmelamine, thiotepa, teniposide, cyclophosphamide, chlorambucil, demethoxyhypocrellin A, melphalan, ifosfamide, trofosfamide, Treosulfan, podophyllotoxin or podophyllotoxin derivatives, etoposide phosphate, teniposide, etoposide, leurosidine, leurosine, vindesine, 9-aminocamptothecin, camptoirinotecan, crisnatol, megestrol, methopterin, mitomycin C, ecteinascidin 743, busulfan, carmustine (BCNU), lomustine (CCNU), lovastatin, 1-methyl-4-phenylpyridinium ion, semustine, staurosporine, streptozocin, phthalocyanine, dacarbazine, aminopterin, methotrexate, trimetrexate, thioguanine, mercaptopurine, fludarabine, pentastatin, cladribin, cytarabine (ara C), porfiromycin, 5-fluorouracil, 6-mercaptopurine, doxorubicin hydrochloride, leucovorin, mycophenolic acid, daunorubicin, deferoxamine, floxuridine, doxifluridine, raltitrexed, idarubicin, epirubican, pirarubican, zorubicin, mitoxantrone, bleomycin sulfate, actinomycin D, safracins, saframycins, quinocarcins, discodermolides, vincristine, vinblastine, vinorelbine tartrate, vertoporfin, paclitaxel, tamoxifen, raloxifene, tiazofuran, thioguanine, ribavirin, EICAR, estramustine, estramustine phosphate sodium, flutamide, bicalutamide, buserelin, leuprolide, pteridines, enediynes, levamisole, aflacon, interferon, interleukins, aldesleukin, filgrastim, sargramostim, rituximab, BCG, tretinoin, betamethasone, gemcitabine hydrochloride, verapamil, VP-16, altretamine, thapsigargin, oxaliplatin, iproplatin, tetraplatin, lobaplatin, DCP, PLD-147, JM118, JM216, JM335, satraplatin, docetaxel, deoxygenated paclitaxel, TL-139, 5′-nor-anhydrovinblastine (hereinafter: 5′-nor-vinblastine), camptothecin, irinotecan (Camptosar, CPT-11), topotecan (Hycamptin), BAY 38-3441, 9-nitrocamptothecin (Orethecin, rubitecan), exatecan (DX-8951), lurtotecan (GI-147211C), gimatecan, homocamptothecins diflomotecan (BN-80915) and 9-aminocamptothecin (IDEC-13′), SN-38, ST1481, karanitecin (BNP1350), indolocarbazoles (e.g., NB-506), protoberberines, intoplicines, idenoisoquinolones, benzo-phenazines or NB-506.

In another related embodiment, the invention contemplates the practice of the method in conjunction with other anti-tumor therapies such as radiation. As used herein, the term “radiation” is intended to include any treatment of a neoplastic cell or subject by photons, neutrons, electrons, or other type of ionizing radiation. Such radiations include, but are not limited to, X-ray, gamma-radiation, or heavy ion particles, such as alpha or beta particles. Additionally, the radiation may be radioactive. The means for irradiating neoplastic cells in a subject are well known in the art and include, for example, external beam therapy, and brachytherapy.

Methods to determine if a cancer (rumor or neoplasia) has been treated are well known to those skilled in the art and include, for example, a decrease in the number of tumor cells (e.g., a decrease in cell proliferation or a decrease in tumor size). It is recognized that the treatment of the present invention may be a lasting and complete response or can encompass a partial or transient clinical response. See for example, Isselbacher et al. (1996) Harrison's Principles of Internal Medicine 13 ed., 1814-1882, incorporated herein by reference.

Assays to test for the sensitization or the enhanced death of tumor cells are well known in the art, including, for example, standard dose response assays that assess cell viability; agarose gel electrophoresis of DNA extractions or flow cytometry to determine DNA fragmentation, a characteristic of cell death; assays that measure the activity of polypeptides involved in apoptosis; and assay for morphological signs of cell death. The details regarding such assays are described elsewhere herein. Other assays include, chromatin assays (e.g., counting the frequency of condensed nuclear chromatin) or drug resistance assays as described in, for example, Lowe et al. (1993) Cell 74:95 7-697, herein incorporated by reference. See also U.S. Pat. No. 5,821,072, also herein incorporated by reference.

### Selective Cell Killing

The invention provides compounds that kill cancer cells and methods for killing such cells, especially genotype-specific cancer cells, such as those with elevated Ras signaling activity. Such methods can be performed either in vivo, ex vivo or in vitro. One aspect of the invention provides a method to selectively kill cancer cells, especially those with elevated Ras activity, the method comprising administering to a mammalian patient in need of treatment a therapeutically effective amount of a compound disclosed herein.

In certain embodiments, the invention provides a method of selectively killing cancer cells that have elevated Ras pathway activity while protecting relatively normal cells that do not have elevated Ras activity. This can be useful since many cancers harbor the somatic RasV12 or other similar mutations leading to elevated Ras signaling activity in cancer cells, while normal cells in the same patient/individual usually do not have the same RasV12 or other Ras pathway mutations. Compounds of the invention can be used to selectively kill these cancer cells. The subject method would be effective in killing cancer cells since normal cells likely do not have elevated Ras signaling activity.

In yet other embodiments, cells could be sensitized to a compound of the invention through the introduction or expression of a target protein or proteins. Expression can be accomplished by infection of target cells with vectors, such as adenoviral or retroviral vectors expressing the target protein (see below).

Alternatively, the target protein may be directly provided to the target cells. For example, the protein(s) may be introduced into the target cells using various methods known in the art (see details below). In one embodiment, the protein may be provided to the target cell by entrapping it in liposomes bearing positive charges on their surface (e.g., lipofectins) and which are optionally tagged with antibodies against cell surface antigens of the target tissue, e.g., antibodies against a cancer cell surface antigen. In another embodiment, the protein may be provided to the target cells by transcytosis, using any of the “internalizing peptides” capable of mediating this effect, including but not limited to the N-terminal domain of the HIV protein Tat (e.g., residues 1-72 of Tat or a smaller fragment thereof which can promote transcytosis), all or a portion of the Drosophila antennopedia III protein, a sufficient portion of mastoparan, etc. (see below).

In other embodiments, the diminished protein (and/or other other target proteins) may be achieved by delivering an antibody, RNAi (siRNA, short hairpin RNA, etc.), antisense sequence, or small molecule inhibitor specific for such target protein.

Delivery of such antagonists of a protein to a target cell is well known in the art. See, for example, WO04078940A2, EP1439227A1, WO04048545A2, US20040029275A1, WO03076592A2, WO04076674A1, WO9746671A1, all incorporated herein by reference.

Another aspect of the invention provides a conjoint therapeutic method using compounds of the invention and one or more agents or therapies (e.g., radiotherapy) that kill cells via an apoptotic mechanism. Such agents include many of the chemotherapeutic drugs described below.

It is believed that certain proteins have elevated expression levels in cells sensitive to compounds of the invention.

In certain embodiments, target cells are manipulated to express a higher level of a target protein(s) so as to enhance the susceptibility of killing or slowing the rate of proliferation by compounds of the invention.

For example, a target protein may be introduced into the target cells using various methods known in the art (see details below). In one embodiment, the target protein may be provided to the target cell by entrapping it in liposomes bearing positive charges on their surface (e.g., lipofectins) and which are optionally tagged with antibodies against cell surface antigens of the target tissue, e.g., antibodies against a cancer cell surface antigen.

Alternatively, nucleic acids encoding a functional target may be introduced into such target cells, using, for example, adenoviral or retroviral vectors.

In addition, endogenous target protein activity may be stimulated by an agent that either stimulates expression, or suppresses the activity of a target protein inhibitor (transcription or translation inhibitor, or inhibitor that promotes protein turnover in the cell).

In certain aspects, the method of the invention also involves administering an agent that increases the abundance of target protein in the cell. The agent for increasing the abundance of target protein can, for example, include a polynucleotide encoding the protein adapted to be transported into the cell, e.g., fused with a heterologous internalization domain or formulated in liposome preparation.

In certain aspects, the method of the invention also involves administering an agent that decreases the abundance of the target protein in the cell. The agent for decreasing the abundance of the target protein can, for example, inhibit endogenous protein expression, suppress protein expression or enhance the function of a protein inhibitor.

### Pharmaceutical Compositions

Prospective therapeutic agents can be profiled in order to determine their suitability for inclusion in a pharmaceutical composition. One common measure for such agents is the therapeutic index, which is the ratio of the therapeutic dose to a toxic dose. The thresholds for therapeutic dose (efficacy) and toxic dose can be adjusted as appropriate (e.g., the necessity of a therapeutic response or the need to minimize a toxic response). For example, a therapeutic dose can be the therapeutically effective amount of an agent (relative to treating one or more conditions) and a toxic dose can be a dose that causes death (e.g., an LD₅₀) or causes an undesired effect in a proportion of the treated population. Preferably, the therapeutic index of an agent is at least 2, more preferably at least 5, and even more preferably at least 10. Profiling a therapeutic agent can also include measuring the pharmacokinetics of the agent, to determine its bioavailability and/or absorption when administered in various formulations and/or via various routes.

A compound of the present invention can be administered to an individual in need thereof. In certain embodiments, the individual is a mammal such as a human, or a non-human mammal. When administered to an individual, the compound of the invention can be administered as a pharmaceutical composition containing, for example, the compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil or injectable organic esters. In a preferred embodiment, when such pharmaceutical compositions are for human administration, the aqueous solution is pyrogen free, or substantially pyrogen free. The excipients can be chosen, for example, to effect delayed release of an agent or to selectively target one or more cells, tissues or organs.

A pharmaceutically acceptable carrier can contain physiologically acceptable agents that act, for example, to stabilize or to increase the absorption of a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The pharmaceutical composition (preparation) also can be a liposome or other polymer matrix, which can have incorporated therein, for example, a compound of the invention. Liposomes, for example, which consist of phospholipids or other lipids, are nontoxic, physiologically acceptable and metabolizable carriers that are relatively simple to make and administer.

A pharmaceutical composition (preparation) containing a compound of the invention can be administered to a subject by any of a number of routes of administration including, for example, orally; intramuscularly; intravenously; anally; vaginally; parenterally; nasally; intraperitoneally; subcutaneously; and topically. The composition can be administered by injection or by incubation. One preferred route, especially for Compound 5 of Group I, is as a sterile, aqueous intravenous solution. Another preferred route, especially for Compound 5 of Group I, is orally as a capsule. In order to improve solubility, compounds of the invention are present as salts (e.g., a hydrochloride salt such as the dihydrochloride salt of Compound 5 of Group I).

In certain embodiments, the compound of the present invention may be used alone or conjointly administered with another type of anti-tumor therapeutic agent. As used herein, the phrase “conjoint administration” refers to any form of administration in combination of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. Thus, an individual who receives such treatment can benefit from a combined effect of different therapeutic compounds.

It is contemplated that the compound of the present invention will be administered to a subject (e.g., a mammal, preferably a human) in a therapeutically effective amount (dose). By “therapeutically effective amount” is meant the concentration of a compound that is sufficient to elicit the desired therapeutic effect (e.g., treatment of a condition, the death of a neoplastic cell). It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. Typically, for a human subject, an effective amount will range from about 0.001 mg/kg of body weight to about 50 mg/kg of body weight. A larger total dose can be delivered by multiple administrations of the agent. Methods to determine efficacy and dosage are known to those skilled in the art. See, for example, Isselbacher et al. (1996) Harrison's Principles of Internal Medicine 13 ed., 1814-1882, herein incorporated by reference. In certain embodiments, the dose is from 4-44 mg/m², such as 8-44 mg/m² or 8-22 mg/m².

In certain embodiments, a compound of the present invention is administered intravenously. In certain such embodiments, the compound is administered via infusion for a period of 15 minutes to 4 hours, such as 30 minutes to 2 hours or 45 to 75 minutes (e.g., about one hour).

In certain embodiments, a compound of the present invention is administered orally (e.g., a capsule). Typically, oral administration occurs once or twice daily (preferably once) for a desired period of time, such as 2-8 weeks (e.g., 2-6 weeks, 2-4 weeks, about 3 weeks).

### EXEMPLIFICATION

The invention now being generally described, it will be more readily understood by reference to the following examples, which are included merely for purposes of illustration of certain aspects and embodiments of the present invention, and are not intended to limit the invention.

### Example 1

The in vitro anti-multiple myeloma (MM) activity of Compound 5 of Group I was evaluated (by MTT survival assays): 34 of 46 MM cell lines (74%) responded to 48 hr treatment with sub-uM concentrations of Compound 5 (achievable in preclinical pharmacokinetic studies). (24 of these lines had IC₅₀ values <300 nM). Activity of the compound was not restricted to MM cells with known Ras mutations.

Importantly, Compound 5 was active against MM cells resistant to conventional (Dex, alkylators, anthracyclines) and/or novel (e.g. lenalidomide, CC-4047, bortezomib, multitargeted kinase inhibitors) anti-MM treatments. Cell death commitment assays revealed that a pharmacologically relevant 5 hr pulse with 300 nM Compound 5 is sufficient to commit MM-1S, NCI-H929 and OPM-2 MM cells to cell death. Importantly, co-culture with BMSCs did not protect MM cells against Compound 5 (at doses nontoxic to BMSCs), indicating that Compound 5 overcomes the protective effect of IL-6. This conclusion is further supported by INA-6 cells being one of the most sensitive cells lines (IC₅₀=100 nM).

Gene expression profiles (with Affymetrix U133 2.0plus oligonucleotide microarrays) showed early (<2 hr) of compound-induced modulation of broad spectrum of genes involved in regulation of cellular bioenergetics. The in vivo anti-MM activity of Compound 5 was evaluated in SCID-beige mice sublethally irradiated with 300 rad, subsequently injected i.v. with 1×106 OPM-2 MM cells (which led to diffuse medullary and extramedullary lesions). Mice were randomly assigned to receive, by oral gavage, either Compound 5 100 mg/kg (n=14) or vehicle only (n=14), on a cyclical schedule of 5 days-on/2 days-off treatment. After 47 days of oral administration of Compound 5, median overall survival had not been reached in the Compound 5-treated cohort (12/14 mice still alive, one lost to the tumor, one lost to bacterial infection) vs. 35 days (95% CI: 23-47 days) in the control group (0/14 alive at day 47) (Kaplan-Meier analysis, p<0.0001, by log-rank test). Additional results are shown in the figures.

Based upon these results, Compound 5 represents a promising novel orally bioavailable agent for advanced MM patients.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1-16. (canceled)

17. A method of treating resistant multiple myeloma, comprising administering to a patient in need thereof a therapeutically effective amount of an erastin analog, wherein the erastin analog is a compound represented by structural formula (V): 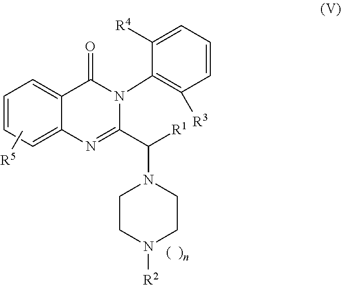
or a pharmaceutically acceptable salt thereof, wherein
R¹ is H or C₁₋₈ alkyl;
R² is H or C₁₋₈ alkyl;
R³ is halogen, C₁₋₈ alkoxy, or C₁₋₈ alkyl;
R⁴ is H, halogen, C₁₋₈ alkoxy, or C₁₋₈ alkyl;
R⁵ is H, halogen, or nitro; and
n is 1 or 2,
wherein the multiple myeloma is resistant to one or more of dexamethasone, alkylators, anthracyclines, doxorubicin, lenalidomide, bortezomib, and multitargeted kinase inhibitors.

18. A method of treating multiple myeloma characterized by a cell type selected from the group consisting of one or more OPM-2 cells, MM-1S cells, MM-1R cells, KMS-18 cells, S6B45 cells, MR20 cells and/or ARD cells, comprising administering to a patient in need thereof a therapeutically effective amount of an erastin analog, wherein the erastin analog is a compound represented by structural formula (V): 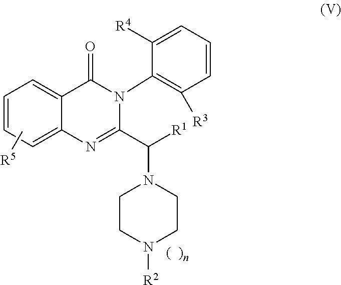
or a pharmaceutically acceptable salt thereof, wherein
R¹ is H or C₁₋₈ alkyl;
R² is H or C₁₋₈ alkyl;
R³ is halogen, C₁₋₈ alkoxy, or C₁₋₈ alkyl;
R⁴ is H, halogen, C₁₋₈ alkoxy, or C₁₋₈ alkyl;
R⁵ is H, halogen, or nitro; and
n is 1 or 2.

19. The method of claim 17, wherein the compound is represented by the following formula:

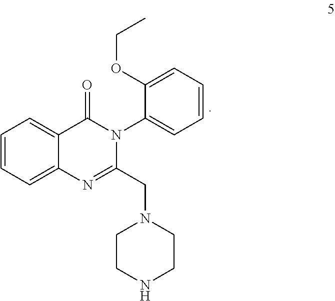 [SMILES: CCOc1ccccc1-n1c(CN2CCNCC2)nc2ccccc2c1=O]

20. The method of claim 17, further comprising conjointly administering to said patient an agent that kills cells through an apoptotic mechanism.

21. The method of claim 20, wherein said agent is a chemotherapeutic agent.

22. The method of claim 21, wherein said chemotherapeutic agent is selected from the group consisting of: an EGF-receptor antagonist, arsenic sulfide, adriamycin, cisplatin, carboplatin, cimetidine, carminomycin, mechlorethamine hydrochloride, pentamethylmelamine, thiotepa, teniposide, cyclophosphamide, chlorambucil, demethoxyhypocrellin A, melphalan, ifosfamide, trofosfamide, Treosulfan, podophyllotoxin or podophyllotoxin derivatives, etoposide phosphate, teniposide, etoposide, leurosidine, leurosine, vindesine, 9-aminocamptothecin, camptoirinotecan, crisnatol, megestrol, methopterin, mitomycin C, ecteinascidin 743, busulfan, carmustine, lomustine, lovastatin, 1-methyl-4-phenylpyridinium ion, semustine, staurosporine, streptozocin, phthalocyanine, dacarbazine, aminopterin, methotrexate, trimetrexate, thioguanine, mercaptopurine, fludarabine, pentastatin, cladribin, cytarabine, porfiromycin, 5-fluorouracil, 6-mercaptopurine, doxorubicin hydrochloride, leucovorin, mycophenolic acid, daunorubicin, deferoxamine, floxuridine, doxifluridine, raltitrexed, idarubicin, epirubican, pirarubican, zorubicin, mitoxantrone, bleomycin sulfate, actinomycin D, safracins, saframycins, quinocarcins, discodermolides, vincristine, vinblastine, vinorelbine tartrate, vertoporfin, paclitaxel, tamoxifen, raloxifene, tiazofuran, thioguanine, ribavirin, 5-ethynyl-1-beta-D-ribofuranosylimidazole-carboxamide (ElGAR), estramustine, estramustine phosphate sodium, flutamide, bicalutamide, buserelin, leuprolide, pteridines, enediynes, levamisole, aflacon, interferon, interleukins, aldesleukin, filgrastim, sargramostim, rituximab, bacillus Calmette-Guerin (BCG), tretinoin, betamethasone, gemcitabine hydrochloride, verapamil, etoposide phosphate (VP-16), altretamine, thapsigargin, oxaliplatin, iproplatin, tetraplatin, lobaplatin, cis-dichlorobis(cyclopentylamine)platinum(ll) (DCP), bis(acetato)(1-adamantylamine)ammine-dichloro-platinum (IV) (PLD-147), amminedichloro(cyclohexylamine) platinum (II) (JM118), bis-acetato-ammine-dichloro-cyclohexylamine-platinum (IV) (JM216), trans-ammine-(cyclohexylamine)-dichloro-dihydroxo-platinum (IV) (JM335), satraplatin, docetaxel, deoxygenated paclitaxel, milataxel (TL-139), 5′-nor-anhydrovinblastine, camptothecin, irinotecan, topotecan, afeletecan (BAY 38-3441), 9-nitrocamptothecin, exatecan, lurtotecan, gimatecan, homocamptothecins diflomotecan and 9-aminocamptothecin, 7-ethyl-10-hydroxy-camptothecin (SN-38), gimatecan (ST 1481), karanitecin, indolocarbazoles, protoberberines, intoplicines, idenoisoquinolones, benzo-phenazines, and N-formylamino-12,13-dihydro-1,11-dihydroxy-13-(13-d-glucopyranosyi)-5H-indolo[2,3-a]pyrrolo[3,4-c]carbazole-5,7(6H)-dione (NB-506).

23. The method of claim 17, wherein the erastin analog is, 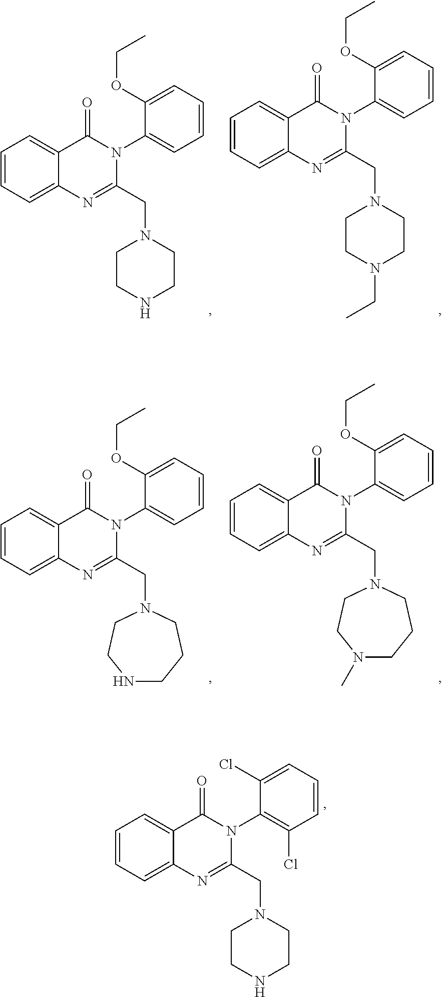
or a pharmaceutically acceptable salt thereof.

24. The method of claim 17, wherein the multiple myeloma is resistant to one or more of anthracyclines, lenalidomide, bortezomib, and multitargeted kinase inhibitors.

25. The method of claim 17, wherein the multiple myeloma comprises cells that have at least one Ras mutation, wherein the at least one Ras mutation is not a K-Ras mutation.

26. The method of claim 17, wherein the multiple myeloma comprises cells that have at least one Ras mutation, wherein the at least one Ras mutation is not a K-Ras or an N-Ras mutation.

27. The method of claim 17, wherein the multiple myeloma comprises cells that do not have a Ras mutation.

28. The method of claim 18, wherein the multiple myeloma is characterized by OPM-2 cells.

29. The method of claim 18, wherein the compound is represented by the following formula:

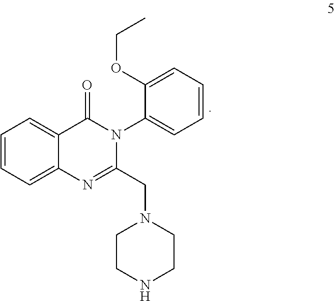 [SMILES: CCOc1ccccc1-n1c(CN2CCNCC2)nc2ccccc2c1=O]

30. A method of treating resistant multiple myeloma, comprising administering to a patient in need thereof a therapeutically effective amount of an erastin analog, wherein the erastin analog is a compound represented by structural formula (V): 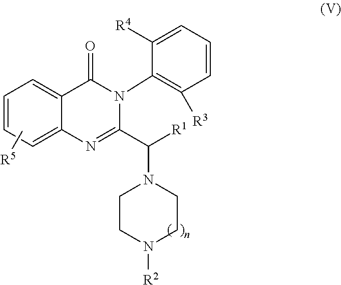
or a pharmaceutically acceptable salt thereof, wherein
R¹ is H or C₁₋₈ alkyl;
R² is H or C₁₋₈ alkyl;
R³ is halogen, C₁₋₈ alkoxy, or C₁₋₈ alkyl;
R⁴ is H, halogen, C₁₋₈ alkoxy, or C₁₋₈ alkyl;
R⁵ is H, halogen, or nitro; and
n is 1 or 2,
wherein the multiple myeloma is resistant to one or more of dexamethasone, alkylators, anthracyclines, doxorubicin, lenalidomide, bortezomib, and multitargeted kinase inhibitors,
and wherein the multiple myeloma is characterized by a cell type selected from the group consisting of one or more OPM-2 cells, MM-1S cells, MM-1R cells, KMS-18 cells, S6B45 cells, MR20 cells and/or ARD cells.

31. The method of claim 30, wherein the multiple myeloma is resistant to one or more of anthracyclines, lenalidomide, bortezomib, and multitargeted kinase inhibitors.

32. The method of claim 30, wherein the multiple myeloma comprises cells that have at least one Ras mutation, wherein the at least one Ras mutation is not a K-Ras mutation.

33. The method of claim 30, wherein the multiple myeloma comprises cells that have at least one Ras mutation, wherein the at least one Ras mutation is not a K-Ras or an N-Ras mutation.

34. The method of claim 30, wherein the multiple myeloma comprises cells that do not have a Ras mutation.

35. The method of claim 30, wherein the multiple myeloma is characterized by OPM-2 cells.

36. The method of claim 30, wherein the compound is represented by the following formula: 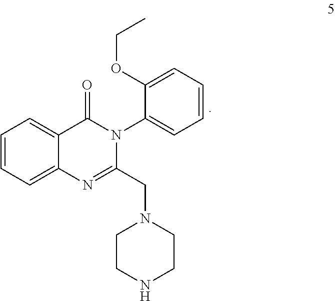 [SMILES: CCOc1ccccc1-n1c(CN2CCNCC2)nc2ccccc2c1=O]
